(19) United States
(12) Patent Application Publication
(10) Pub. No.: US 2006/0057151 A1
(43) Pub. Date: Mar. 16, 2006

(54) BmpB novel nucleotide and amino acid sequences and diagnostic and therapeutic uses thereof

(76) Inventors: David John Hampson, Mt. Nasura (AU); Tom La, Beckenham (AU)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/195,063

(22) Filed: Aug. 2, 2005

Related U.S. Application Data
(62) Division of application No. 10/739,583, filed on Dec. 18, 2003.

(30) Foreign Application Priority Data
Dec. 19, 2002 (AU) 2002953431

(51) Int. Cl. A61K 39/00; A61K 39/38

(52) U.S. Cl. 424/184.1

(57) ABSTRACT

An isolated amino acid sequence comprising the sequence set out in SEQ ID NO:2 or an amino acid sequence substantially homologous thereto, or a fragment thereof, with the proviso that the amino acid sequence in SEQ ID NO:3 is specifically excluded.

### STATEMENT OF RELATED APPLICATIONS

The present application claims the benefit of priority under 35 U.S.C. § 119 of Australian Application No. 2002953431, which was filed Dec. 19, 2002 and is incorporated herein by reference in its entirety.

### FIELD OF THE INVENTION

The present invention relates generally to the field of swine dysentery. Specifically, the invention relates to a novel Brachyspira hyodysenteriae amino acid sequence encoding an outer membrane lipoprotein BmpB and the polynucleotide sequence that encodes it. More specifically, the invention relates to the use of these sequences for the prophylactic and therapeutic treatment, including vaccines, for swine dysentery. The invention also relates to the diagnosis of the presence of B. hyodysenteriae. The invention further relates to the screening of drugs for swine dysentery therapy. Finally, the invention relates to prophylactic, therapeutic and diagnostic compositions derived from the nucleotide and amino acid sequences described herein.

### BACKGROUND ART

Swine dysentery is a significant endemic disease of pigs in Australia and worldwide. Swine dysentery is a contagious mucohaemorrhagic diarrhoeal disease, characterised by extensive inflammation and necrosis of the epithelial surface of the large intestine. Economic losses due to swine dysentery result mainly from growth retardation, costs of medication and mortality. The causative agent of swine dysentery was first identified as an anaerobic spirochaete (Treponema hyodysenteriae) in 1971, and was recently reassigned to the genus Brachyspira as B. hyodysenteriae. Where swine dysentery is established in a piggery, the disease spectrum can vary from being mild, transient or unapparent to being severe and even fatal. Medication strategies on individual piggeries may mask clinical signs and on some piggeries the disease may go unnoticed, or may only be suspected. Whether or not obvious disease occurs, B. hyodysenteriae may persist in infected pigs, or in other reservoir hosts such as rodents, or in the environment. All these sources pose potential for transmission of the disease to uninfected herds.

Colonisation by B. hyodysenteriae elicits a strong immunological response against the spirochaete, hence indirect evidence of exposure to the spirochaete can be obtained by measuring circulating antibody titres in the blood of infected animals. These antibody titres have been reported to be maintained at low levels, even in animals that have recovered from swine dysentery. Serological tests for detection of antibodies therefore have considerable potential for detecting subclinical infections and recovered carrier pigs that have undetectable numbers of spirochaetes in their large intestines. These tests would be particularly valuable in an easy to use kit form, such as an enzyme-linked immunosorbent assay. A variety of techniques have been developed to demonstrate the presence of circulating antibodies against B. hyodysenteriae, including indirect fluorescent antibody tests, haemagglutination tests, microtitration agglutination tests, complement fixation tests, and ELISA using either lipopolysaccharide or whole sonicated spirochaetes as antigen. All these tests have suffered from problems of specificity, as related non-pathogenic intestinal spirochaetes can induce cross-reactive antibodies. These tests are useful for detecting herds where there is obvious disease and high circulating antibody titres, but they are problematic for identifying sub-clinically infected herds and individual infected pigs. Consequently, to date, no completely sensitive and specific assays are available for the detection of antibodies against B. hyodysenteriae. The lack of suitable diagnostic tests has hampered control of swine dysentery.

A number of methods are employed to control swine dysentery, varying from the prophylactic use of antimicrobial agents, to complete destocking of infected herds and prevention of re-entry of infected carrier pigs. All these options are expensive and, if they are to be fully effective, they require the use of sophisticated diagnostic tests to monitor progress. Currently, detection of swine dysentery herds with sub-clinical infections, and individual healthy carrier animals, remains a major problem and is hampering implementation of effective control measures. A definitive diagnosis of swine dysentery traditionally has required the isolation and identification of B. hyodysenteriae from the faeces or mucosa of diseased pigs. Major problems involved include the slow growth and fastidious nutritional requirements of these anaerobic bacteria and confusion due to the presence of morphologically similar spirochaetes in the normal flora of the pig intestine. A significant improvement in the diagnosis of individual affected pigs was achieved with the development of polymerase chain reaction (PCR) assays for the detection of spirochaetes from faeces. Unfortunately in practical applications the limit of detection of PCRs rendered it unable to detect carrier animals with subclinical infections. As a consequence of these diagnostic problems, there is a clear need to develop a simple and effective diagnostic tool capable of detecting B. hyodysenteriae infection at the herd and individual pig level.

A strong immunological response is induced against the spirochaete following colonization with B. hyodysenteriae, and pigs recovered from SD are protected from re-infection. Despite this, attempts to develop vaccines to control SD have met with very limited success, either because they have provided inadequate protection on a herd basis, or they have been too costly and difficult to produce to make them commercially viable. Bacterin vaccines provide some level of protection, but they tend to be lipopolysaccharide serogroup-specific, which then requires the use of multivalent bacterins. Furthermore they are difficult and costly to produce on a large scale because of the fastidious anaerobic growth requirements of the spirochaete.

Several attempts have been made to develop attenuated live vaccines for SD. This approach has the disadvantage that attenuated strains show reduced colonisation, and hence cause reduced immune stimulation. There also is a reluctance on the part of producers and veterinarians to use live vaccines for SD because of the possibility of reversion to virulence, especially as very little is known about genetic regulation and organization in B. hyodysenteriae.

The use of recombinant subunit vaccines is an attractive alternative, since the products would be well-defined (essential for registration purposes), and relatively easy to produce on a large scale. To date the only reported use of a recombinant protein from B. hyodysenteriae as a vaccine candidate (a 38-kilodalton flagellar protein) failed to prevent colonisation in pigs. This failure is likely to relate specifically to the particular recombinant protein used, as well as to other more down-stream issues of delivery systems and routes, dose rates, choice of adjuvants etc. A number of attempts have been made to identify outer envelop proteins from B. hyodysenteriae that could be used as recombinant vaccine components, but again no successful vaccine has yet been made. A much more global approach is needed to the identification of potentially useful immunogenic recombinant proteins from B. hyodysenteriae is needed.

The present invention provides a novel B. hyodysenteriae amino acid sequence and the polynucleotide sequence that encodes it, which has not previously been identified.

### SUMMARY OF THE INVENTION

We have identified a novel amino acid sequence, referred to herein as Brachyspira membrane protein B (BmpB), as well as amino acid fragments thereof that are particularly suited to diagnostic, prophylactic and therapeutic purposes associated with swine dysentery. We have also identified the polynucleotide sequence encoding the BmpB amino acid sequence.

Accordingly, the present invention provides a BmpB amino acid sequence which comprises the sequence set out in SEQ ID NO:2 or an amino acid sequence substantially homologous thereto, or a fragment of the amino acid sequence of SEQ ID NO:2, with the proviso that the amino acid sequence in SEQ ID NO:3 is specifically excluded from the invention. In one preferred embodiment of the invention there are provided fragments of the BmpB amino acid sequence, which fragments are selected from SEQ ID NO:4 to SEQ ID NO:17.

The invention also provides a BmpB polynucleotide sequence (SEQ ID NO:1) or a homologue thereof. Preferably, the BmpB polynucleotide sequence is selected from: (a) polynucleotide sequences comprising the nucleotide sequence set out in SEQ ID NO:1 or a fragment thereof; (b) polynucleotide sequences comprising a nucleotide sequence capable of selectively hybridising to the polynucleotide sequence set out in SEQ ID NO:1 or a fragment thereof; (c) polynucleotide sequences that are degenerate, as a result of the genetic code, to the sequences defined in (a) or (b), or (d) Polynucleotide sequences complementary to the sequences of (a), (b) or (c).

Detectably labelled nucleotide sequences hybridisable to a polynucleotide sequence of the invention are also provided and include nucleotide sequences hybridisable to a coding or non-coding region of a BmpB polynucleotide sequence. The present invention also provides oligonucleotide primers for amplifying B. hyodysenteriae genomic DNA encoding a BmpB amino acid sequence such as set out in SEQ ID NOS:2 and 4 through 17.

Vectors provided by the invention will contain a BmpB polynucleotide sequence according to the invention. Preferably, the vectors are either cloning or expression vectors. Where the vector is an expression vector, it preferentially comprises a BmpB polynucleotide sequence operatively associated with an expression control sequence.

Also provided are unicellular cells transformed or transfected with a polynucleotide sequence of the invention or with a vector as described above. Preferred cells include: bacteria, yeast, mammalian cells, plant cells, insect cells, or swine cells in tissue culture.

The invention further provides methods for preparing a BmpB amino acid sequence comprising: (a) culturing a cell as described above under conditions that provide for expression of a BmpB amino acid sequence; and (b) recovering the expressed BmpB amino acid sequence. This procedure can also be accompanied by the steps of: (c) chromatographing the amino acid sequence on a Ni-chelation column; and (d) purifying the amino acid sequence by gel filtration.

The invention also provides labelled and unlabelled monoclonal and polyclonal antibodies or fragments or recombinant derivatives thereof that are specific for a BmpB amino acid sequence of the invention and immortal cell lines that produce a monoclonal antibody of the invention. Antibody preparation according to the invention involves: (a) conjugating a BmpB amino acid sequence to a carrier protein; (b) immunising a host animal with the BmpB amino acid sequence fragment-carrier protein conjugate of step (a) admixed with an adjuvant; and (c) obtaining BmpB specific antibody from the immunised host animal.

The invention further provides a method for detecting the presence or absence of B. hyodysenteriae in a biological sample, which method comprises: (a) bringing the biological sample into contact with a polynucleotide probe or primer comprising a BmpB polynucleotide sequence of the invention under suitable hybridising conditions; and (b) detecting any duplexes formed between the probe or primer and the nucleotide sequences in the sample.

The invention provides methods for measuring the presence of a BmpB amino add sequence in a sample, comprising: (a) contacting a sample suspected of containing a BmpB amino acid sequence with an antibody that specifically binds to the BmpB amino acid sequence under conditions which allow for the formation of a reaction complex; and (b) detecting the formation of the reaction complex, wherein detection of the formation of a reaction complex indicates the presence of a BmpB amino add sequence in the sample.

The invention also provides a method for detecting swine dysentery antibodies in biological samples, which comprises: (a) providing a BmpB amino acid sequence or a fragment thereof; (b) incubating a biological sample with said amino acid sequence under conditions which allow for the formation of an antibody antigen complex; and (c) detecting said antibody-antigen complex

Correspondingly provided are in vitro methods for evaluating the level of BmpB amino add sequence in a biological sample comprising: (a) detecting the formation of reaction complexes in a biological sample according to the method noted above; and (b) evaluating the amount of reaction complexes formed, which amount corresponds to the level of BmpB amino acid sequence in the biological sample. Further, there are provided in vitro methods for monitoring therapeutic treatment of a disease associated B. hyodysenteriae in an animal host comprising evaluating, as describe above, the levels of BmpB amino acid sequence in a series of biological samples obtained at different time points from an animal host undergoing such therapeutic treatment.

The invention also addresses the use of polynucleotide sequences of the invention, as well as antisense nucleic acid sequences hybridisable to a polynucleotide encoding a BmpB amino acid sequence according to the invention, for the manufacture of a medicament for modulation of a disease associated with B. hyodysenteriae.

Additionally, the invention provides pharmaceutical or therapeutic compositions or agents including, but not limited to vaccines for the prevention, amelioration or treatment of SD associated with B. hyodysenteriae, comprising: (a) at least a BmpB amino acid sequence, as described herein or at least a BmpB nucleotide sequence as described herein or an antibody that specifically bind to one of the aforementioned sequences; and (b) one or more pharmaceutically acceptable carriers and/or diluents.

The invention further provides a polynucleotide, amino acid sequence and/or antibody of the invention for use in therapy. Also provided is a method of treating a condition characterised by swine dysentery, which method comprises administering to an animal in need of treatment an effective amount of a polynucleotide, amino acid sequence or antibody of the invention. Further, the invention provides a method for prophylactically treating an animal to prevent or at least minimise swine dysentery, comprising the step of: administering to the animal an effective amount of a polynucleotide, polypeptide, an antibody or a pharmaceutical composition comprising one or more of these biological molecules.

In addition, the invention provides methods of screening drugs capable of modulating the biological activity of B. hyodysenteriae through either direct or indirect interaction with a BmpB nucleotide or amino acid sequence. A substance identified by these methods may be used in a method of treating swine dysentery.

The invention also provides kits for screening animals suspected of being infected with B. hyodysenteriae or to confirm that an animal is infected with B. hyodysenteriae, which kits comprise at least a polynucleotide complementary to a portion of the BmpB polynucleotide sequence, packaged in a suitable container, together with instructions for its use in an alternate farm, the invention provides kits for (a) screening host animals suspected of being infected with B. hyodysenteriae, or (b) to confirm that a host animal is infected with B. hyodysenteriae, which kits comprise at least a BmpB amino acid sequence or fragment thereof or an antibody which binds the aforementioned sequences packaged in a suitable container and instructions for its use.

Other aspects and advantages of the invention will become apparent to those skilled in the art from a review of the ensuing description, which proceeds with reference to the following illustrative drawings.

### BRIEF DESCRIPTION OF DRAWINGS

FIG. 1 represents the BmpB polynucleotide and amino acid sequence.

FIG. 2 represents the Western blot of recombinant truncated BmpB with the Anti-Histidine monoclonal antibody. Lane 1=molecular weight marker, lane 2=BmpB-F13/R809, lane 3=BmpB-F13/R195, lane 4=BmpB-F13/R411, lane 5=BmpB-F13/R613. Molecular weight marker was BenchMark pre-stained protein marker (Life Technologies).

FIG. 3 represents the Western blot of recombinant truncated BmpB with the monoclonal antibody BJL/SH1. Lane 1=molecular weight marker, lane 2=BmpB-F13/R809, lane 3=BmpB-F13/R195, lane 4=BmpB-F13/R411, lane 5=BmpB-F13/R613. Molecular weight marker was pre-stained low range protein marker. (Biorad).

FIG. 4 shows a graphical representation of the BmpB-F604/R809 ELISA results. The threshold value was defined as two standard deviations above the mean of the OD values obtained from the healthy pigs. Thirteen naturally infected pigs (•), 9 healthy pigs (∘), and 21 swine dysentery outbreak pigs (▾).

FIG. 5 shows a graphical representation of the systemic antibody titres (ELISA) of the unvaccinated and vaccinated pigs directed against recombinant BmpB before and after challenge with B. hyodysenteriae. Circulating antibodies were detected by ELISA using BmpB as the coating antigen.

FIG. 6 shows a graphical representation of the systemic antibody titres (ELISA) of the unvaccinated and vaccinated pigs directly against B. hyodysenteriae whole cell components before and after challenge. Circulating antibodies were detected by ELISA using sonicated and clarified B. hyodysenteriae (homologous strain to infection) as the coating antigen.

FIG. 7 shows a graphical representation of the colonic antibody titres (ELISA) following vaccination of pigs with recombinant BmpB, and following challenge with B. hyodysenteriae. Mucosal antibodies were detected by ELISA using recombinant BmpB and sonicated B. hyodysenteriae whole-cells (same strain as infection) as the coating antigen.

FIG. 8 shows a graphical representation of systemic antibody titres (ELISA) of the control pigs of Group A that were not vaccinated prior to challenge with B. hyodysenteriae. Circulating antibodies targeting recombinant BmpB were detected by ELISA.

FIG. 9 shows a graphical representation of systemic antibody titres of the pigs of group B that were vaccinated with recombinant BmpB prior to challenge with B. hyodysenteriae. Circulating antibodies targeting recombinant BmpB were detected by ELISA.

FIG. 10 represents a Western blot analysis of pooled serum from the pigs of group B that were vaccinated with recombinant BmpB. Sera from four pigs were pooled for each sample time. The antigen used was a whole-cell extract of the homologous B. hyodysenteriae strain used for challenge. Lane 1, serum from a pig hyper-immunised with a B. hyodysenteriae bacterin (positive control); lanes 2-4, serum taken pre-vaccination; lanes 5-7, serum taken pre-challenge; lanes 8-10, serum taken at post-mortem. Each triplicate includes serum taken from pigs 13-16, pigs 17-20 and pigs 21-24, consecutively. Molecular weight markers are shown in kDa. The native BmpB protein of B. hyodysenteriae is indicated with the arrow.

FIG. 11 shows a graphical representation of systemic antibody titres of the pigs of group C that were vaccinated with recombinant MBP-F604 (MBP fused to the C-terminal portion of BmpB) prior to challenge with B. hyodysenteriae. Circulating antibodies targeting recombinant MBP-F604 were detected by ELISA.

FIG. 12 shows a graphical representation of systemic antibody titres of the pigs of group C that were vaccinated with recombinant MBP-F604 (MBP fused to the C-terminal portion of BmpB) prior to challenge with B. hyodysenteriae. Circulating antibodies targeting recombinant BmpB were detected by ELISA.

FIG. 13 represents a Western blot analysis of pooled serum from the pigs of group C that were vaccinated against MBP-F604. Sera from three pigs which indicated some ELISA reactivity to recombinant BmpB was investigated. The antigen used was a whole-cell extract of the homologous B. hyodysenteriae strain used for challenge. Lane 1, serum from a pig hyper-immunised with a B. hyodysenteriae bacterin (positive control); lanes 2-4, serum taken pre-vaccination; lanes 5-7, serum taken pre-challenge; lanes 8-10, serum taken at post-mortem. Each triplicate includes serum taker; from pig 27, pig 31 and pig 35, consecutively. Molecular weight markers are shown in kDa. The native BmpB protein of B. hyodysenteriae is indicated with the arrow.

FIG. 14 shows a graphical representation of mucosal antibody titres (IgA) in the colon following challenge of all unvaccinated and vaccinated (BmpB and MBP-F-604) pigs. Local antibodies were detected by ELISA using recombinant BmpB as the coating antigen.

FIG. 15 represents a Western blot analysis of mucosal IgA in the colon of selected pigs that showed reactivity to recombinant BmpB in ELISA. The antigen used was a whole-cell extract of the B. hyodysenteriae strain used for challenge. Lane 1, pig 1; lane 2, pig 5; lane 3, pig 10; lane 4, pig 17; lane 5, pig 18; lane 6, pig 22; lane 7, pig 24; lane 8, pig 25; lane 9, pig 26; lane 10, pig 27; lane 11, pig 28. Pigs 1-12 were not vaccinated. Pigs 13-24 were vaccinated with recombinant BmpB. Pigs 25-36 were vaccinated with MBP-F604. Molecular weight markers are shown in kDa. The position of the native BmpB protein is indicated with the arrow.

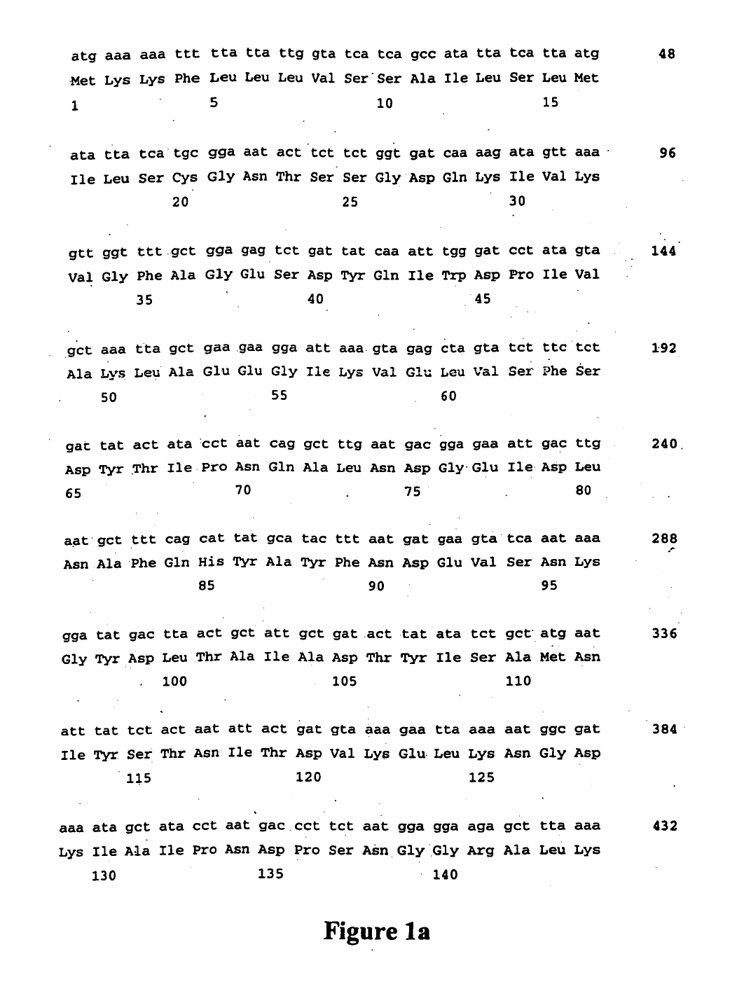

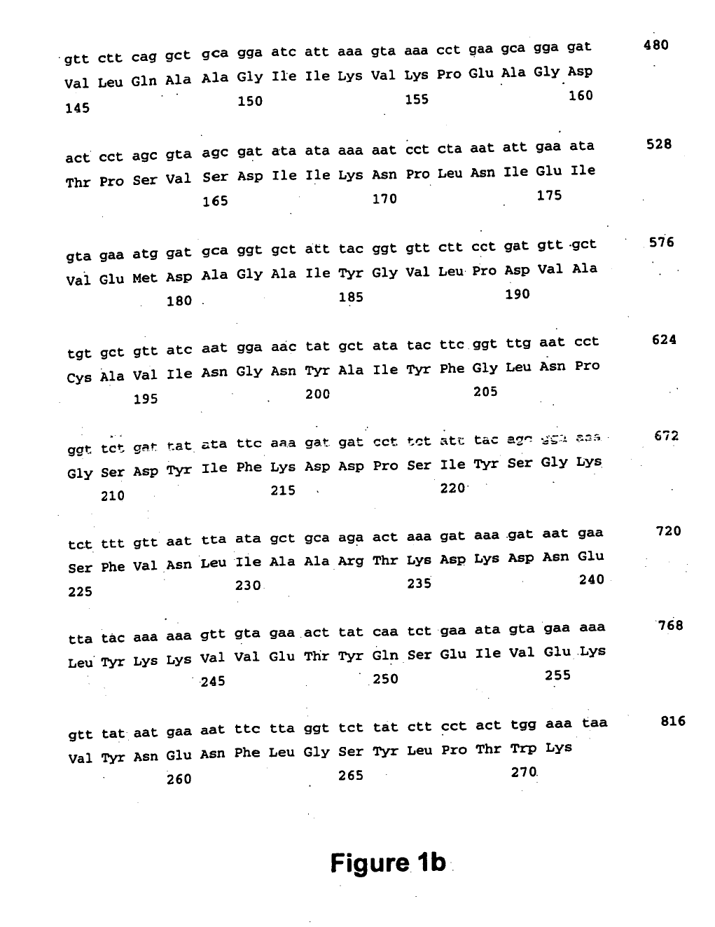

### DETAILED DISCLOSURE OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein. Sequence identity numbers (SEQ ID NO:) containing nucleotide and amino acid sequence information included in this specification are collected at the end of the description and have been prepared using the programme Patentin Version 3.0. Each nucleotide or amino acid sequence is identified in the sequence listing by the numeric indicator <210>followed by the sequence identifier (e.g. <210>1, <210>2, etc.). The length, type of sequence and source organism for each nucleotide or amino acid sequence are indicated by information provided in the numeric indicator fields <211>, <212>and <213>, respectively. Nucleotide and amino acid sequences referred to in the specification are defined by the information provided in numeric indicator field <400>followed by the sequence identifier (e.g. <400>1, <400>2, etc.).

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs

### DESCRIPTION OF PREFERRED EMBODIMENTS

The present invention relates to the identification of BmpB amino acid sequences, including variations and fragments thereof as well as polynucleotide sequences encoding said sequences.

The BmpB amino acid sequence was isolated from B. hyodysenterae by screening a B. hyodysenteriae lambda bacteriophage genomic library. Through this screening process immunopositive phagemids were identified that possessed a gene sequence for a 30 kDa outer envelope protein. Sequencing of the phagemids revealed an 816 bp open-reading frame (ORF).

The translated-BlastP homology search of the BmpB amino acid sequence against the SWISS-PRO, protein database identified 33.9-39.9% homology between BmpB and D-methionine-binding lipoproteins. (MetQ) of other bacteria including Escherichia coli, Haemophilus influenzae, Pasteurella multocida, Salmonella typhimurium, Salmonella typhi, Vibrio cholera and Yersinia pestis (Table 1). Homology (32.1-38.4%) was also seen between BmpB and the gene products (PlpABC) of a tandem multiple gene loci encoding 30 kDa membrane lipoproteins of Pasteurella haemolytica (Table 1). Comparison of the BmpB polynucleotide sequence with the GenBank nucleotide database did-not reveal any strong homology with other bacterial genes.

Sequence homology of the translated BmpB polynucleotide sequence (271 amino acids) with the amino acid sequence of bacterial lipoproteins obtained from the SWISS-PROT protein database is shown in Table 1 below.

TABLE 1

| | | Size | | Homology | Accession |
|---|---|---|---|---|---|
| Organism | Protein | (aa) | Identity (aa) | (%) | Number |
| Salmonella | MetQ | 271 | 108 | 39.9 | Q8ZRN1 |
| typhimurium | | | | | |
| Escherichia | MetQ | 271 | 107 | 39.5 | P28635 |
| coli | | | | | |
| K-12 | | | | | |
| Salmonella | MetQ | 271 | 107 | 39.5 | Q8Z992 |
| typhi | | | | | |
| Escherichia | MetQ | 271 | 106 | 39.1 | Q8X8V9 |
| coli | | | | | |
| O157: H7 | | | | | |
| Yersinia | MetQ | 271 | 105 | 38.7 | Q8ZH40 |
| pestis | | | | | |
| Pasteurella | PlpA | 277 | 87 | 32.1 | Q08868 |
| haemolytica | | | | | |
| | PlpB | 276 | 94 | 34.7 | Q08869 |
| | PlpC | 263 | 104 | 38.4 | Q08870 |
| Vibrio | MetQ | 269 | 99 | 36.5 | Q9KTJ7 |
| cholera | | | | | |
| Haemophilus | MetQ | 273 | 93 | 34.3 | P31728 |
| influenzae | | | | | |
| Pasteurella | MetQ | 276 | 92 | 33.9 | Q9CK95 |
| multocida | | | | | |

Analysis of the BmpB polynucleotide sequence: revealed a potential Shine-Dalgamo ribosome binding site (AGGAG), and putative −10 (TATAAT) and −35 (TTGAAA) promoter regions upstream from the ATG start codon. A 12 bp region with dyad symmetry was present downstream from the TAA stop codon. The BmpB polynucleotide sequence comprises 291 adenosine residues, 278 tyrosine residues (69.7% A/T), 141 guanine residues, and 106 cytosine residues (30.3% G/C), as shown in SEQ ID NO:1.

BmpB Amino Acid Sequences

Full-length BmpB amino acid sequences provided according to the invention will have about 271 amino acids and encode a B. hyodysenteriae outer membrane lipoprotein. Analysis of the BmpB amino acid sequence revealed the presence of a 19 amino acid lipoprotein precursor signal peptide (MKKFLLLVSSAILSLMILS) at the N-terminal of the sequence. A Kyte-Doolittle hydropathy plot of the sequence showed this N-terminal to be highly hydrophobic. The prolipoprotein (272 aa) and mature lipoprotein (253 aa) have predicted molecular masses of 29,682 daltons and 27,593 daltons, respectively.

BmpB amino acid sequences of the invention include those having the amino acid sequence set forth herein e.g., SEQ ID NOS: 2 and 4 through 17. They also include BmpB amino acid sequences modified with conservative amino acid substitutions, as well as analogues, fragments and derivatives thereof, with the proviso that the amino acid sequence in SEQ ID NO:3 is specifically excluded.

The amino acid sequence in SEQ ID NO:3 is specifically excluded from the invention. However, the proviso should not be understood to exclude sequences which include SEQ ID NO:3. That is, BmpB amino acid sequences of the invention having the amino acid sequence of SEQ ID NO:2, SEQ ID NO:4 through to SEQ ID NO:17, as well as analogues, fragments and derivatives thereof, which include the amino acid sequence in SEQ ID NO:3 are not excluded. In addition, the proviso should not be understood to exclude nucleotide sequences which include the nucleotide sequence encoding the amino acid in SEQ ID NO:3.

In a preferred form of the invention there is provided an isolated BmpB amino acid sequence as herein described. More desirably the BmpB amino acid sequence is provided in substantially purified form.

The term “isolated” is used to describe a BmpB amino acid sequence that has been separated from components that accompany it in its natural state. Further, a BmpB amino acid sequence is “substantially purified” when at least about 60 to 75% of a sample exhibits a single BmpB amino acid sequence. A substantially purified BmpB amino acid sequence will typically comprise about 60 to 90% W/W of a BmpB amino acid sequence sample, more usually about 90%, and preferably will be over about 95% pure. Protein, purity or homogeneity may be indicated by a number of means well known in the art, such as polyacrylamide gel electrophoresis of a protein sample, followed by visualizing a single BmpB amino acid sequence band upon staining the gel. For certain purposes, higher resolution may be provided by using HPLC or other means well known in the art which are utilised for application.

Preferred BmpB amino acid sequences of the invention will have one or more biological properties (eg in vivo, in vitro or immunological properties) of the native full-length BmpB amino acid sequence. Non-functional BmpB amino acid sequences are also included within the scope of the invention since they may be useful, for example, as antagonists of BmpB. The biological properties of analogues, fragments, or derivatives relative to wild type may be determined, for example, by means of biological assays.

BmpB amino acid sequences, including analogues, fragments and derivatives, can be prepared synthetically (e.g., using the well known techniques of solid phase or solution phase peptide synthesis). Preferably, solid phase synthetic techniques are employed. Alternatively, BmpB amino acid sequences of the invention can be prepared using well known genetic engineering techniques, as described infra. In yet another embodiment, BmpB amino acid sequences can be purified (e.g., by immunoaffinity purification) from a biological fluid, such as but not limited to plasma, faeces, serum, or urine from swine.

Analogues of the BmpB Amino Acid Sequence

BmpB amino acid sequence analogues include those having the amino acid sequence, wherein one or more of the amino acids are substituted with another amino acid which substitutions do not substantially alter the biological activity of the molecule.

In the context of the invention, an analogous sequence is taken to include a BmpB amino acid sequence which is at least 60, 70, 80 or 90% homologous, preferably at least 95 or 98% homologous at the amino acid level over at least 20, 50, 100 or 200 amino acids, with the amino acid sequences set out in SEQ ID NO:2. In particular, homology should typically be considered with respect to those regions of the sequence known to be essential for the function of the protein rather than non-essential neighbouring sequences. Particularly preferred BmpB amino acid sequences of the invention comprise a contiguous sequence having greater than 60 or 70% homology, more preferably greater than 80 or 90% homology, to one or more of amino acid sequences shown as SEQ ID NO:4 to SEQ ID NO:17.

Although homology can be considered in terms of similarity (i.e. amino add residues having similar chemical properties/functions), in the context of the present invention it is preferred to express homology in terms of sequence identity. The terms “substantial homology” or “substantial identity”, when referring to BmpB amino acid sequences, indicate that the BmpB amino acid sequence in question exhibits at least about 70% identity with an entire naturally-occurring BmpB amino acid sequence or portion thereof, usually at least about 80% identity and preferably at least about 90 or 95% identity.

In a highly preferred form of the invention a BmpB amino acid sequence analogue will have 80% or greater amino acid sequence identity to the BmpB amino acid sequence set out in SEQ ID NO:2 or to a sequence as shown in SEQ ID NO: 4 through SEQ ID NO: 17. Examples of BmpB amino acid sequence analogues within the scope of the invention include the amino acid sequence of SEQ ID NO:2 wherein: (a) one or more aspartic acid residues is substituted with glutamic acid; (b) one or more isoleucine residues is substituted with leucine; (c) one or more glycine or valine residues is substituted with alanine; (d) one or more arginine residues is substituted with histidine; or (e) one or more tyrosine or phenylalanine residues is substituted with tryptophan.

Screening for BmpB Analogues

Various screening techniques are known in the art for screening for analogues of polypeptides. Various libraries of chemicals are available. Accordingly, the present invention contemplates screening such libraries, e.g., libraries of synthetic compounds generated over years of research, libraries of natural compounds and combinatorial libraries, as described in greater detail, infra, for analogues of the BmpB amino acid sequence. In one embodiment, the invention contemplates screening such libraries for analogues that bind to BmpB specific antibodies.

Fragments of the BmpB Amino Acid Sequences

In addition to analogues, the invention contemplates fragments of the BmpB amino acid sequence except for the fragment that is shown in SEQ ID NO:3.

A BmpB amino acid sequence fragment is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids. Preferred BmpB amino acid sequence fragments include those sequences as shown in SEQ ID NO:4 through SEQ ID NO:17, or analogues thereof.

In a highly preferred form of the invention the fragments exhibit ligand-binding, immunological activity and/or other biological activities characteristic of BmpB amino acid sequences. More preferably, the fragments possess immunological epitopes consistent with those present on native BmpB amino acid sequences.

As used herein, “epitope” refers to an antigenic determinant of a polypeptide. An epitope could comprise three amino acids in a spatial conformation that is unique to the epitope. Generally, an epitope consists of at least five amino acids, and more usually consists of at least 8-10 amino acids. Methods of determining the spatial conformation of such amino acids are known in the art.

BmpB Amino Acid Sequence Derivatives

“BmpB amino acid sequence derivatives” are provided by the invention and include BmpB amino acid sequences, analogues or fragments thereof which are substantially homologous in primary structural but which include chemical and/or biochemical modifications or unusual amino acids. Such modifications include, for example, acetylation, carboxylation, phosphorylation, glycosylation, biotinylation, ubiquitination, labeling, (e.g., with radioactive and chemiluminescent nucleotides), and various enzymatic modifications, as will be readily appreciated by those well skilled in the art.

In one form of the invention the chemical moieties suitable for derivatisation are selected from among water soluble polymers. The polymer selected should be water soluble so that the protein to which it is attached does not precipitate in an aqueous environment, such as a physiological environment. Preferably, for therapeutic use of the end-product preparation, the polymer will be pharmaceutically acceptable. One skilled in the art will be able to select the desired polymer based on considerations such as whether the polymer/protein conjugate will be used therapeutically, and if so, the desired dosage, circulation time, resistance to proteolysis and other considerations. For the present proteins and peptides, these may be ascertained using the assays provided herein.

The water soluble polymer may be selected from the group consisting of, for example, polyethylene glycol, copolymers of ethylene glycol propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide co-polymers, polyoxyethylated polyols and polyvinyl alcohol. Polyethylene glycol propionaldenhyde may provide advantages in manufacturing due to its stability in water.

In another form of the invention the amino acid sequences may be modified to produce a longer half life in an animal host, for example, by fusing one or more antibody fragments (such as an Fc fragment) to the amino or carboxyl end of a BmpB amino acid sequence.

Where the BmpB amino acid sequence is to be provided in a labelled form, a variety of methods for labeling amino acid sequences are well known in the art and include radioactive isotopes such as ³²P, ligands which bind to labelled antiligands (eg, antibodies), fluorophores, chemiluminescent agents, enzymes and antiligands which can serve as specific binding pair members for a labelled ligand. The choice of label depends on the sensitivity required, stability requirements, and available instrumentation. Methods of labeling amino acid sequences are well known in the art [See, e.g., Sambrook et al., Molecular Cloning: A Laboratory Manual, Second Edition, Cold Spring Harbor Laboratory Press, Cold Spring Harbor, N.Y. (1989); and Ausubel, F., Brent, R., Kingston, R. E., Moore, D. D., Seidman, J. G., Smith, J. A., Struhl, K. Current protocols in molecular biology. Greene Publishing Associates/Wiley Intersciences, New York (2001)].

The BmpB amino acid sequences of the invention, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

The invention also provides for fusion polypeptides, comprising BmpB amino acid sequences and fragments. Thus BmpB amino acid sequences may be fusions between two or more BmpB amino acid sequences or between a BmpB amino acid sequence and a related protein. Likewise, heterologous fusions may be constructed which would exhibit a combination of properties or activities of the derivative proteins. For example, ligand-binding or other domains may be “swapped” between different fusion polypeptides or fragments. Such homologous or heterologous fusion polypeptides may display, for example, altered strength or specificity of binding. Fusion partners include immunoglobulins, bacterial toxins, bacterial beta-galactosidase, trpE, protein A, beta-lactamase, alpha amylase, alcohol dehydrogenase and yeast alpha mating factor.

Modified BmpB amino acid sequences may be synthesised using conventional techniques, or may be encoded by a modified polynucleotide sequence and produced using recombinant nucleic acid methods. The modified polynucleotide sequence may also be prepared by conventional techniques. Fusion proteins will typically be made by either recombinant nucleic acid methods or may be chemically synthesised.

BmpB Polynucleotides

According to the invention there is provided an isolated or substantially pure BmpB polynucleotide sequence, which encodes a BmpB amino acid sequence, or analogue, fragment, or derivative thereof. Preferred BmpB polynucleotide sequences according to the invention comprise the sequence set out in SEQ ID NO:1 or fragments thereof.

A “BmpB polynucleotide sequence” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single-stranded form, or a double-stranded helix. Double-stranded DNA-DNA, DNA-RNA and RNA-RNA helices are possible. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

An “isolated” or “substantially pure” BmpB polynucleotide is one that is substantially separated from other cellular components that naturally accompany a native B. hyodysenteriae genomic sequence. The term embraces a BmpB polynucleotide sequence that has been removed from its naturally occurring environment and includes recombinant or cloned BmpB polynucleotide sequence isolates and chemically synthesised variants or variants biologically synthesised by heterologous systems.

In one embodiment, the invention provides BmpB polynucleotide sequences for expression of a BmpB amino acid sequence. More specifically, the BmpB polynucleotide sequence is selected from the group consisting of: (a) polynucleotide sequences set out in SEQ ID NO:1 or fragments thereof; (b) polynucleotide sequences that hybridise to the polynucleotide sequence defined in (a) or hybridisable fragments thereof; and (c) polynucleotide sequences that code on expression for the amino acid sequence encoded by any of the foregoing polynucleotide sequences.

Homologous BmPB Polynucleotide Sequences

BmpB polynucleotide sequences of the invention will include a sequence that is either derived from, or substantially similar to a natural BmpB polynucleotide sequence or one having substantial homology with a natural BmpB polynucleotide sequence or a portion thereof. A BmpB polynucleotide sequence is “substantially homologous”: (“or substantially similar”) to another if, when optimally aligned (with appropriate nucleotide insertions or deletions) with the other polynucleotide sequence (or its complementary strand), there is nucleotide sequence identity in at least about 60% of the nucleotide bases, usually at least about 70%, more usually at least about 80%, preferably at least about 90% and more preferably at least about 95-98% of the nucleotide bases.

Alternatively, substantial homology or identity exists when a BmpB polynucleotide sequence or fragment thereof will hybridise to another BmpB polynucleotide (or a complementary strand thereof under selective hybridisation conditions, to a strand, or to its complement. Typically, selective hybridisation will occur when there is at least about 55% identity over a stretch of at least about 14 nucleotides, preferably at least about 65%, more preferably at least about 75% and most preferably at least about 90%. The length of homology comparison, as described, may be over longer stretches and in certain embodiments will often be over a stretch of at least about nine nucleotides, usually at least about; 20 nucleotides, more usually at least about 24 nucleotides, typically at least about 28 nucleotides, more typically at least about 32 nucleotides and preferably at least about 36 or more nucleotides.

Thus, the polynucleotide sequences of the invention preferably have at least 75%, more preferably at least 85%, more preferably at least 90% homology to the sequences shown in the sequence listings herein. More preferably there is at least 95%, more preferably at least 98%, homology. Nucleotide homology comparisons may be conducted as described below for polypeptides. A preferred sequence comparison program is the GCG Wisconsin Bestfit program.

In the context of the present invention, a homologous sequence is taken to include a nucleotide sequence which is at least 60, 70, 80 or 90% identical, preferably at least 95 or 98% identical at the nucleic acid level over at least 20, 50, 100, 200, 300, 500 or 819 nucleotides with the nucleotides sequences set out in SEQ ID NO:1. In particular, homology should typically be considered with respect to those regions of the sequence that encode contiguous amino acid sequences known to be essential for the function of the protein rather than non-essential neighbouring sequences.

Other preferred BmpB polynucleotide sequences of the invention comprise a contiguous sequence having greater than 50 , 60 or 70% homology, more preferably greater than 80, 90, 95 or 97% homology, to the nucleotide sequence that encodes one or more of the amino acid sequences of SEQ ID NO:4 to SEQ ID NO:17.

BmpB Polynucleotide Sequence Fragments

BmpB polynucleotide sequence fragments of the invention will preferably be at least 15 nucleotides in length, more preferably at least 20, 30, 40, 50, 100 or 200 nucleotides in length. Generally, the shorter the length of the polynucleotide sequence, the greater the homology required to obtain selective hybridisation. Consequently, where a polynucleotide sequence of the invention consists of less than about 30 nucleotides, it is preferred that the percentage identity is greater than 75%, preferably greater than 90% or 95% compared with the polynucleotide sequences set out in the sequence listings herein. Conversely, where a polynucleotide sequence of the invention consists of, for example, greater than 50 or 100 nucleotides, the percentage identity compared with the polynucleotide sequences set out in the sequence listings herein may be lower, for example greater than 50%, preferably greater than 60 or 75%.

BmpB Probe Sequences

Contemplated within the scope of the present invention are probe sequences derived from BmpB polynucleotide sequences, which can be conveniently prepared from the specific sequences disclosed herein. Probes may be of any suitable length, which span all or a portion of the BmpB polynucleotide sequence and which allow specific hybridisation to that sequence.

The greater the degree of homology, the more stringent the hybridisation conditions that can be used. Thus, in one embodiment, preferably the probes are designed so that low stringency hybridisation conditions are used to identify homologous BmpB polynucleotide sequences. In an alternate embodiment the probes are designed such that moderate hybridisation conditions are used. More preferably highly stringent conditions are used. As demonstrated experimentally herein, a BmpB probe sequence will hybridise to a polynucleotide sequence such as depicted in SEQ ID NO:1 under moderately stringent conditions; more preferably, it will hybridise under high stringency conditions.

Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC 0.15 M NaCl, 0.015 M sodium citrate pH 7.0).

Preferably, the probe sequences will have a nucleotide sequence of at least about eight consecutive nucleotides, from SEQ ID NO:1, or preferably about 15 consecutive nucleotides, or more preferably at least about 25 nucleotides, and may have a minimal size of at least about 40 nucleotides. Particularly preferred, oligonucleotide probes for detecting BmpB polynucleotide sequences include the oligonucleotide sequences set out in SEQ ID NO:18 to SEQ ID NO:25 and in the Examples.

The probes of the invention may include an isolated polynucleotide attached to a label or reporter molecule and may be used to isolate other polynucleotide sequences, having sequence similarity by standard methods. For techniques for preparing and labeling probes see, e.g. Sambrook et al., (1989) supra or Ausubel et al., (2001) supra.

Probes comprising synthetic oligonucleotides or other polynucleotide sequences of the present invention may also be derived from naturally occurring or recombinant single- or double-stranded polynucleotides, or be chemically synthesised. Probes may be labelled by nick translation, Klenow fill-in reaction, or other methods known in the art.

BmpB Primer Sequences

The present invention also provides BmpB primer sequences. Primers employed in amplification reactions are preferably single stranded for maximum efficiency in amplification, but may be double stranded. If double stranded, primers may be first treated to separate the strands before being used to prepare extension products. Primers should be sufficiently long to prime the synthesis of BmpB extension products in the presence of the inducing agent for polymerisation. The exact length of a primer will depend on many factors, including temperature, buffer, and nucleotide composition.

Oligonucleotide primers will typically contain 12-20 or more nucleotides, although they may contain fewer nucleotides. Preferably, the primers are selected from the sequences depicted in SEQ ID NO: 18 to SEQ ID NO: 25.

Oligonucleotide primers may be prepared using any suitable method, such as conventional phosphotriester and phosphodiester methods or automated embodiments thereof. In one such automated embodiment, diethylphosphoramidites are used as starting materials and may be synthesized as described by Beaucage, et al., (1981) Tetrahedron Letters, 22:1859-1862. One method for synthesising oligonucleotides on a modified solid support is described in U.S. Pat. No. 4,458,066.

Antisense Nucleic Adds and Ribozymes

The present invention also extends to the preparation of antisense nucleotides and ribozymes that may be used to interfere with the expression of BmpB amino acid sequences at the translational level. This approach utilises antisense nucleic acid and ribozymes to block translation of a specific mRNA, either by masking that mRNA with an antisense nucleic acid or cleaving it with a ribozyme.

Antisense nucleic acids are DNA or RNA molecules that are complementary to at least a portion of a specific mRNA molecule [See: Weintraub, (1990) Sd. Am., 262:4046; Marcus-Sekura, (1988) Anal. Biochem., 172:289-295]. In the cell, they hybridise to that mRNA, forming a double-stranded molecule. The cell does not translate an mRNA complexed in this double-stranded form. Therefore, antisense nucleic acids interfere with the expression of mRNA into protein. Oligomers of about fifteen nucleotides and molecules that hybridise to the AUG initiation codon will be particularly efficient, since they are easy to synthesize and are likely to pose fewer problems than larger molecules when introducing them into infected cells. Antisense methods have been used to inhibit the expression of many genes in vitro [Hambor et al., (1988) J. Exp. Med., 168:1237-1245].

Ribozymes are RNA molecules possessing the ability to specifically cleave other single-stranded RNA molecules in a manner somewhat analogous to DNA restriction endonucleases. Ribozymes were discovered from the observation that certain mRNAs have the ability to excise their own introns. By modifying the nucleotide sequence of these RNAs, researchers have been able to engineer molecules that recognise specific nucleotide sequences in an RNA molecule and cleave it [Cech, (1988) J. Am. Med. Assoc., 260:3030-3034]. Because they are sequence-specific, only mRNAs with particular sequences are inactivated.

Investigators have identified two types of ribozymes, Tetrahymena-type and “hammerhead”-type. Tetrahymena-type ribozymes recognize four-base sequences, while “hammerhead”-type recognize eleven- to eighteen-base sequences. The longer the recognition sequence, the more likely it is to occur exclusively In the target mRNA species. Therefore, hammerhead-type ribozymes are preferable to Tetrahymena-type ribozymes for inactivating a specific mRNA species and eighteen base recognition sequences are preferable to shorter recognition sequences.

The BmpB polynucleotide sequences described herein may thus be used to prepare antisense molecules against and ribozymes that cleave mRNAs for BmpB amino acid sequences, thus inhibiting expression of the BmpB polynucleotide sequences.

Isolation of BmpB Polynucleotide Sequences

Any B. hyodysenteriae specimen, in purified or non-purified form, can be utilised as the starting point for the isolation of BmpB polynucleotide sequences. Such specimens are preferentially extracted from a swine sample, such as blood, tissue material or faeces and the like by a variety of techniques such as those described by Maniatis, et. al. in Molecular Cloning: A Laboratory Manual, Cold Spring Harbor, N.Y., p 280-281, (1982).

If the extracted sample has not been purified, it may be treated before isolation of the BmpB polynucleotide sequence with an amount of a reagent effective to open the cells, or bacterial cell membranes of the sample and to expose and/or separate the strand(s) of the nucleic acid(s).

Once B. hyodysenteriae genomic material has been liberalised there are a number of methods by which a BmpB polynucleotide sequence may be amplified and/or isolated. Details of such methods may be derived from Sambrook et al., (1989) supra or Ausubel et al., (2001) supra.

PCR is perhaps one of the more common approaches that may be used to initially amplify BmpB polynucleotide sequences and is preferably used in the invention. Specific BmpB polynucleotide sequences to be amplified may be a fraction of a larger molecule or can be present initially as a discrete molecule, so that the specific sequence constitutes the entire nucleic acid. It is not necessary that the sequence to be amplified is present initially in a pure form; it may be a minor fraction of a complex mixture, such as contained in whole B. hyodysenteriae DNA.

According to the PCR process, deoxyribonucleotide triphosphates dATP, dCTP, dGTP and dTTP are added to the synthesis mixture, either separately or together with the primers, in adequate amounts and the resulting solution is heated to about 90° C.-100° C. from about 1 to 10 minutes, preferably from 1 to 4 minutes. After this heating period, the solution is allowed to cool, which is preferable for the primer hybridisation. To the cooled mixture is added an appropriate agent for effecting the primer extension reaction (called herein “agent for polymerisation”), and the reaction is allowed to occur under conditions known in the art. The agent for polymerisation may also be added together with the other reagents if it is heat stable. This synthesis (or amplification) reaction may occur at room temperature up to a temperature above, which the agent for polymerisation no longer functions. Thus, for example, if DNA polymerase is used as the agent, the temperature is generally no greater than about 40° C. Most conveniently the reaction occurs at room temperature.

The newly synthesised BmpB strand and its complementary nucleic acid strand will form a double-stranded molecule under hybridising conditions described above and this hybrid is used in subsequent steps of the process.

The steps of denaturing, annealing, and extension product synthesis can be repeated as often as needed to amplify the target BmpB polynucleotide sequence to the extent necessary for detection. The amount of the specific BmpB polynucleotide sequence produced will accumulate in an exponential fashion. Such amplification reactions are described in more detail in PCR. A Practical Approach, ILR Press, Eds. M. J. McPherson, P. Quirke, and G. R. Taylor, 1992.

The BmpB polynucleotide amplification products may be detected by Southern blots analysis, without using radioactive probes. In such a process, for example, a small sample of DNA containing a very low level of the nucleic acid sequence of the BmpB polynucleotide sequence is amplified and analysed via a Southern blotting technique or similarly, using dot blot analysis. The use of non-radioactive probes or labels is facilitated by the high level of the amplified signal. Alternatively, probes used to detect the amplified products can be directly or indirectly detectably labelled, as described herein.

Sequences amplified by the methods of the invention can be further evaluated, detected, cloned, sequenced and the like, either in solution or after binding to a solid support, by any method usually applied to the detection of a specific DNA sequence such as PCR, oligomer restriction [Saiki, et. al. (1985), Bio/Technology, 3:1008-1012), allele-specific oligonucleotide (ASO) probe analysis [Conner, et. al., (1983) Proc. Natl. Acad. Sci. U.S.A., 80:278], oligonucleotide ligation assays (OLAs) [Landgren, et. al. (1988), Science, 241:1007], and the like.

Alternative methods of amplification have been described and can also be employed in the invention. Such alternative amplification systems include but are not limited to self-sustained sequence replication and nucleic acid sequence-based amplification (which uses reverse transcription and T7 RNA polymerase and incorporates two primers to target its cycling scheme). Alternatively, BmpB polynucleotide sequence can be amplified by ligation activated transcription or a ligase chain reaction or the repair chain reaction nucleic acid amplification technique.

BmpB Polynucleotide Constructs and Vectors

According to another embodiment the present invention provides methods for preparing a BmpB amino acid sequence, comprising the steps of: (a) culturing a cell as described herein under conditions that provide for expression of the BmpB amino acid sequence; and (b) recovering the expressed BmpB sequence. This procedure can also be accompanied by the steps of: (c) chromatographing the amino acid sequence using any suitable means known in the art; and/or (d) subjecting the amino acid sequence to protein purification.

To produce a cell capable of expressing BmpB amino acid sequences, preferably polynucleotide sequences of the invention are incorporated into a recombinant vector, which is then introduced into a host prokaryotic or eukaryotic cell.

Vectors provided by the present invention will typically comprise a BmpB polynucleotide sequence encoding the desired amino acid sequence and preferably transcription and translational initiation regulatory sequences operably linked to the amino acid encoding sequence. Examples of such expression vectors are described in Sambrook et al., (1989) supra or Ausubel et al., (2001) supra. Many useful vectors are known in the art and may be obtained from such vendors as Stratagene, New England Biolabs, Promega Biotech, and others.

Expression vectors may also include, for example, an origin of replication or autonomously replicating sequence and expression control sequences, a promoter, an enhancer and necessary processing information sites, such as ribosome-binding sites, RNA splice sites, polyadenylation sites, transcriptional terminator sequences, and mRNA stabilising sequences. Secretion signals may also be included where appropriate, from secreted polypeptides of the same or related species, which allow the protein to cross and/or lodge in cell membranes, and thus attain its functional topology, or to be secreted from the cell. Such vectors may be prepared by means of standard recombinant techniques well known in the art and discussed, for example, in Sambrook et al., (1989) supra or Ausubel et al., (2001) supra.

An appropriate promoter and other necessary vector sequences will be selected so as to be functional in the host, and may include, when appropriate, those naturally associated with outer membrane lipoprotein genes.

Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Vectors and promoters suitable for use in yeast expression are further described in Hitzeman et al., EP 73,675A. Appropriate non-native mammalian promoters might include the early and late promoters from SV40 or promoters derived from murine Moloney leukaemia virus, avian sarcoma viruses, adenovirus II, bovine papilloma virus or polyoma. In addition, the construct may be Joined to an amplifiable gene (e.g., DHFR) so that multiple copies of the gene may be made. For appropriate enhancer and other expression control sequences.

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

Vectors containing BmpB polynucleotide sequences can be transcribed in vitro and the resulting RNA introduced into the host cell by well-known methods, e.g. by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of BmpB polynucleotide sequences into the host cell may be achieved by any method known in the art, including, inter alia, those described above.

The invention also provides host cells transformed or transfected with a BmpB polynucleotide sequence. Preferred host cells include yeast, filamentous fungi, plant cells, insect, amphibian, avian species, bacteria, mammalian cells, and human cells in tissue culture. Illustratively, such host cells are selected from the group consisting of E. coli, Pseudomonas, Bacillus, Streptomyces, yeast, CHO, R1.1, B-W, L-M, COS 1, COS 7, BSC1, BSC40, BMT10, and Sf9 cells.

Large quantities of BmpB polynucleotide sequence of the invention may be prepared by expressing BmpB polynucleotide sequences or portions thereof in vectors or other expression vehicles in compatible prokaryotic or eucaryotic host cells. The most commonly used prokaryotic hosts are strains of Escherichia coli, although other prokaryotes, such as Bacillus subtilis or Pseudomonas may also be used. Examples of commonly used mammalian host cell lines are VERO and HeLa cells, Chinese hamster ovary (CHO) cells, and WI38, BHK, and COS cell lines, although it will be appreciated by the skilled practitioner that other cell lines may be appropriate.

Also provided are mammalian cells containing a BmpB polynucleotide sequences modified in vitro to permit higher expression of BmpB amino acid sequence by means of a homologous recombinational event consisting of inserting an expression regulatory sequence in functional proximity to the BmpB amino acid sequence encoding sequence.

Antibodies to the BmpB Amino Acid Sequence

According to the invention, BmpB amino acid sequences produced recombinantly or by chemical synthesis and fragments or other derivatives or analogues thereof, including fusion proteins, may be used as an immunogen to generate antibodies that recognize the BmpB amino acid sequence. Such antibodies include but are not limited to polyclonal, monoclonal, chimeric, single chain, Fab fragments and a Fab expression library.

A molecule is “antigenic” when it is capable of specifically interacting with an antigen recognition molecule of the immune system, such as an immunoglobulin (antibody) or T cell antigen receptor. An antigenic amino acid sequence contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic need not be itself immunogenic, i.e., capable of eliciting an immune response without a carrier.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567, as well as antigen binding portions of antibodies, including Fab, F(ab′)₂ and F(v) (including single chain antibodies). Accordingly, the phrase “antibody molecule” in its various grammatical forms as used herein contemplates both an intact immunoglobulin molecule and an immunologically active portion of an immunoglobulin molecule containing the antibody combining site. An “antibody combining site” is that structural portion of an antibody molecule comprised of heavy and light chain variable and hypervariable regions that specifically binds antigen.

Exemplary antibody molecules are intact immunoglobulin molecules, substantially intact immunoglobulin molecules and those portions of an immunoglobulin molecule that contain the paratope, including those portions known in the art as Fab, Fab′, F(ab′)₂ and F(v), which portions are preferred for use in the therapeutic methods described herein.

Fab and F(ab′)₂ portions of antibody molecules are prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction with mercaptoethanol of the disulfide bonds linking the two heavy chain portions, and followed by alkylation of the resulting protein mercaptan with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The term “adjuvant” refers to a compound or mixture that enhances the immune response to an antigen. An adjuvant can serve as a tissue depot that slowly releases the antigen and also as a lymphoid system activator that non-specifically enhances the immune response [Hood et al., in Immunology, p. 384, Second Ed., Benjamin/Cummings, Menlo Park, Calif. (1984)]. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil, or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

Various procedures known in the art may be used for the production of polyclonal antibodies to BmpB amino acid sequences, or fragment, derivative or analogues thereof. For the production of antibody, various host animals can be immunised by injection with the BmpB amino acid sequence, or a derivative (e.g., fragment or fusion protein) thereof, including but not limited to rabbits, mice, rats, sheep, goats, etc. In one embodiment, the BmpB amino acid sequences or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the BmpB amino acid sequences, or fragments, analogues, or derivatives thereof, any technique that provides for the production of antibody molecules by continuous cell lines in culture may be used. These include but are not limited to the hybridoma technique originally developed by Kohler et al., (1975) Nature, 256:495497, the trioma technique, the human B-cell hybridoma technique [Kozbor et al., (1983) Immunology Today, 4:72], and the EBV-hybridoma technique to produce human monoclonal antibodies [Cole et al., (1985) in Monoclonal Antibodies and Cancer Therapy, pp. 77-96, Alan R. Liss, Inc.]. Immortal, antibody-producing cell lines can be created by techniques other than fusion, such as direct transformation of B lymphocytes with oncogenic DNA, or transfection with Epstein-Barr virus. See, e.g., U.S. Pat. Nos. 4,341,761; 4,399,121; 4,427,783; 4,444,887; 4,451,570; 4,466,917; 4,472,500; 4,491,632; and 4,493,890.

In an additional embodiment of the invention, monoclonal antibodies can be produced in germ-free animals utilising recent technology. According to the invention, swine antibodies may be used and can be obtained by using swine hybridomas or by transforming swine B cells with EBV virus in vitro. In fact, according to the invention, techniques developed for the production of “chimeric antibodies” [Morrison et al., (1984) J. Bacteriol., 159-870; Neuberger et al., (1984) Nature, 312:604-608; Takeda et al., (1985) Nature, 314:452-454] by splicing the genes from a mouse antibody molecule specific for an BmpB amino acid sequence together with genes from a swine antibody molecule of appropriate biological activity can be used; such antibodies are within the scope of this invention. Such swine chimeric antibodies are preferred for use in therapy of swine diseases or disorders. (described infra), since the swine antibodies are much less likely than xenogenic antibodies to induce an immune response, in particular an allergic response, themselves.

According to the invention, techniques described for the production of single chain antibodies (U.S. Pat. No. 4,946,778) can be adapted to produce BmpB amino acid sequence-specific single chain antibodies. An additional embodiment of the invention utilises the techniques described for the construction of Fab expression libraries [Huse et al., (1989) Science, 246:1275-1281] to allow rapid and easy identification of monoclonal Fab fragments with the desired specificity for an BmpB amino acid sequence, or its derivatives, or analogues.

Antibody fragments, which contain the idiotype of the antibody molecule, can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA, “sandwich” immunoassays, immunoradiometric assays, gel diffusion precipitin reactions, immunodiffusion assays, in situ immunoassay (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. For example, to select antibodies that recognise a specific epitope of a BmpB amino acid sequence, one may assay generated hybridomas for a product that binds to a BmpB amino acid sequence fragment containing such epitope. For selection of an antibody specific to a BmpB amino acid sequence from a particular species of animal, one can select on the basis of positive binding with BmpB amino acid sequence expressed by or isolated from cells of that species of animal.

Diagnosis

In accordance with another embodiment the invention provides diagnostic and prognostic methods to detect the presence of B. hyodysenteriae using BmpB amino acid sequences and/or antibodies derived there from and/or BmpB polynucleotide sequences.

Diagnostic and prognostic methods will generally be conducted using a biological sample obtained from a swine. A “sample” refers to a sample of tissue or fluid suspected of containing a B. hyodysenteriae polynucleotide or polypeptide from a swine, but not limited to, e.g., plasma, serum, faecal samples, tissue and samples of in vitro cell culture constituents.

Polypeptide/Antibody-Based Diagnostics

The invention provides methods for detecting the presence of an BmpB amino acid sequence in a sample, comprising: (a) contacting a sample suspected of containing an BmpB amino acid sequence with an antibody (preferably bound to a solid support) that specifically binds to the BmpB amino acid sequence under conditions which allow for the formation of reaction complexes comprising the antibody and the BmpB amino acid sequence; and (b) detecting the formation of reaction complexes comprising the antibody and BmpB amino acid sequence in the sample, wherein detection of the formation of reaction complexes indicates the presence of BmpB, amino acid sequence in the sample.

Preferably, the antibody used in this method is derived from an affinity-purified polyclonal antibody, and more preferably a mAb. In addition, it is preferable for the antibody molecules used herein be in the form of Fab, Fab′, F(ab′)₂ or F(v) portions or whole antibody molecules.

Particulary preferred methods for detecting B. hyodysenteriae based on the above method include enzyme linked immunosorbent assays, radioimmunoassays, immunoradiometric assays and immunoenzymatic assays, including sandwich assays using monoclonal and/or polyclonal antibodies.

Three such procedures that are especially useful utilise either the BmpB amino acid sequence (or a fragment thereof) labelled with a detectable label, antibody Ab1 labelled with a detectable label, or antibody Ab₂ labelled with a detectable label. The procedures may be summarized by the following equations wherein the asterisk indicates that the particle is labelled and “AA” stands for the BmpB amino acid sequence:
AA*+Ab₁=AA*Ab₁ A.
AA+Ab*₁=AA Ab₁* B.
AA+Ab₁+Ab₂*=Ab₁AA Ab₂* C.
The procedures and their application are al familiar to those skilled in the art and accordingly may be utilised within the scope of the present invention. The “competitive” procedure, Procedure A, is described in U.S. Pat. Nos. 3,654,090 and 3,850,752. Procedure B is representative of well-known competitive assay techniques. Procedure C, the “sandwich” procedure, is described in U.S. Patent Nos. RE 31,006 and 4,016,043. Still other procedures are known, such as the “double antibody” or “DASP” procedure.

In each instance, the BmpB amino acid sequences form complexes with one or more antibody(ies) or binding partners and one member of the complex is labelled with a detectable label. The fact that a complex has formed and, if desired, the amount thereof, can be determined by known methods applicable to the detection of labels.

It will be seen from the above, that a characteristic property of Ab₂ is that it will react with Ab₁. This is because Ab₁, raised in one mammalian species, has been used in another species as an antigen to raise the antibody, Ab₂. For example, Ab₂ may be raised in goats using rabbit antibodies as antigens. Ab₂ therefore would be anti-rabbit antibody raised in goats. For purposes of this description and claims, Ab₁ will be referred to as a primary antibody, and Ab₂ will be referred to as a secondary or anti-Ab1 antibody.

The labels most commonly employed for these studies are radioactive elements, enzymes, chemicals that fluoresce when exposed to ultraviolet light, and others.

A number of fluorescent materials are known and can be utilised as labels. These include, for example, fluorescein, rhodamine and auramine. A particular detecting material is anti-rabbit antibody prepared in goats and conjugated with fluorescein through an isothiocyanate.

The BmpB amino acid sequence or their binding partners can also be labelled with a radioactive element or with an enzyme. The radioactive label can be detected by any of the currently available counting procedures. The prefswred isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes, which can be used in these procedures, are known and can be utilized. The preferred enzymes are peroxidase, β-glucuronidase, β-D-glucosidase, β-D-galactosidase, urease, glucose oxidase plus peroxidase and alkaline phosphatase. U.S. Pat. Nos. 3,654,090, 3,850,752 and 4,016,043 are referred to by way of example for their disclosure of alternate labeling material and methods.

The invention also provides a method of detecting swine dysentery antibodies in biological samples, which comprises: (a) providing a BmpB amino acid sequence or a fragment thereof; (b) incubating a biological sample with said amino add sequence under conditions which allow for the formation of an antibody-antigen complex; and (c) determining whether an antibody-antigen complex comprising said amino acid sequence is formed.

In another embodiment of the invention there are provided in vitro methods for evaluating the level of BmpB antibodies in a biological sample comprising: (a) detecting the formation of reaction complexes in a biological sample according to the method noted above; and (b) evaluating the amount of reaction complexes formed, which amount of reaction complexes corresponds to the level of BmpB antibodies in the biological sample.

Further there are provided in vitro methods for monitoring therapeutic treatment of a disease associated B. hyodysenteriae in an animal host comprising evaluating, as describe above, the levels of BmpB antibodies in a series of biological samples obtained at different time points from an animal host undergoing such therapeutic treatment.

Nucleic Acid-Based Diagnostics

The present invention further provides methods for detecting the presence or absence of B. hyodysenteriae in a biological sample, which comprise the steps of: (a) bringing the biological sample into contact with a polynucleotide probe or primer comprising a BmpB polynucleotide of the invention under suitable hybridising conditions; and (b) detecting any duplex formed between the probe or primer and nucleic acid in the sample.

According to one embodiment of the invention, detection of B. hyodysenteriae may be accomplished by directly amplifying BmpB polynucleotide sequences from biological sample, using known techniques and then detecting the presence of BmpB polynucleotide sequences.

In one form of the invention, the target nucleic acid sequence is amplified by PCR and then detected using any of the specific methods mentioned above. Other useful diagnostic techniques for detecting the presence of BmpB polynucleotide sequences include, but are not limited to: 1) allele-specific PCR; 2) single stranded conformation analysis; 3) denaturing gradient gel electrophoresis; 4) RNase protection assays; 5) the use of proteins which recognize nucleotide mismatches, such as the E. coli mutS protein; 6) allele-specific oligonucleotides; and 7) fluorescent in situ hybridisation.

In addition to the above methods BmpB polynucleotide sequences may be detected using conventional probe technology. When probes are used to detect the presence of the BmpB polynucleotide sequences, the biological sample to be analysed, such as blood or serum, may be treated, if desired, to extract the nucleic acids. The sample polynucleotide sequences may be prepared in various ways to facilitate detection of the target sequence; e.g. denaturation, restriction digestion, electrophoresis or dot blotting. The targeted region of the sample polynucleotide sequence usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Sample polynucleotide sequences and probes are incubated under conditions that promote stable hybrid formation of the target sequence in the probe with the putative BmpB polynucleotide sequence in the sample. Preferably, high stringency conditions are used in order to prevent false positives.

Detection, if any, of the resulting hybrid is usually accomplished by the use of labelled probes. Alternatively, the probe may be unlabelled, but may be detectable by specific binding with a ligand that is labelled, either directly or indirectly. Suitable labels and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies and the like. Variations of this basic scheme are known in the art, and include those variations that facilitate separation of the hybrids to be detected from extraneous materials and/or that amplify the signal from the labelled moiety.

It is also contemplated within the scope of this invention that the nucleic acid probe assays of this invention may employ a cocktail of nucleic acid probes capable of detecting BmpB polynucleotide sequences. Thus, in one example to detect the presence of BmpB polynucleotide sequences in a cell sample, more than one probe complementary to BmpB polynucleotide sequences is employed and in particular the number of different probes is alternatively 2, 3, or 5 different nucleic acid probe sequences.

Nucleic acid arrays—DNA Chip-Technology

BmpB polynucleotide sequences (preferably in the form of probes) may also be immobilised to a solid phase support for the detection of B. hyodysenteriae. Alternatively the BmpB polynucleotide sequences will form part of a library of DNA molecules that may be used to detect simultaneously a number of different genes from B. hyodysenteriae. In a further alternate form of the invention BmpB polynucleotide sequences together with other polynucleotide sequences (such as from other bacteria or viruses) may be immobilised on a solid support in such a manner permitting identification of the presence of B. hyodysenteriae and/or any of the other polynucleotide sequences bound onto the solid support.

Techniques for producing immobilised libraries of DNA molecules have been described in the art. Generally, most prior art methods describe the synthesis of single-stranded nucleic acid molecule libraries, using for example masking techniques to build up various permutations of sequences at the various discrete positions on the solid substrate. U.S. Pat. No. 5,837,832 describes an improved method for producing DNA arrays immobilised to silicon substrates based on very large scale integration technology. In particular, U.S. Pat. No. 5,837,832 describes a strategy called “tiling” to synthesize specific sets of probes at spatially defined locations on a substrate that may be used to produce the immobilised DNA libraries of the present invention. U.S. Pat. No. 5,837,832 also provides references for earlier techniques that may also be used. Thus polynucleotide sequence probes may be synthesised in situ on the surface of the substrate.

Alternatively, single-stranded molecules may be synthesised off the solid substrate and each preformed sequence applied to a discrete position on the solid substrate. For example, polynucleotide sequences may be printed directly onto the substrate using robotic devices equipped with either pins or pizo electric devices.

The library sequences are typically immobilised onto or in discrete regions of a solid substrate. The substrate may be porous to allow immobilisation within the substrate or substantially non-porous, in which case the library sequences are typically immobilised on the surface of the substrate. The solid substrate may be made of any material to which polypeptides can bind, either directly or indirectly. Examples of suitable solid substrates include flat glass, silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes may also be mounted on a more, robust solid surface such as glass. The surfaces may optionally be coated with a layer of metal, such as gold, platinum or other transition metal. A particular example of a suitable solid substrate is the commercially available BiaCore™ chip (Pharmacia Biosensors).

Preferably, the solid substrate is generally a material having a rigid or semi-rigid surface. In preferred embodiments, at least one surface of the substrate will be substantially flat, although in some embodiments it may be desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 dots/cm⁻².

The solid substrate is conveniently divided up into sections. This may be achieved by techniques such as photoetching, or by the application of hydrophobic inks, for example teflon-based inks (Cel-line, USA).

Discrete positions, in which each different member of the library is located may have any convenient shape, e.g., circular, rectangular, elliptical, wedge-shaped, etc.

Attachment of the polynucleotide sequences to the substrate may be by covalent or non-covalent means. The polynucleotide sequences may be attached to the substrate via a layer of molecules to which the library sequences bind. For example, the polynucleotide sequences may be labelled with biotin and the substrate coated with avidin and/or streptavidin. A convenient feature of using biotinylated polynucleotide sequences is that the efficiency of coupling to the solid substrate can be determined easily. Since the polynucleotide sequences may bind only poorly to some solid substrates, it is often necessary to provide a chemical interface between the solid substrate (such as in the case of glass) and the nucleic acid sequences. Examples of suitable chemical interfaces include hexaethylene glycol. Another example is the use of polylysine coated glass, the polylysine then being chemically modified using standard procedures to introduce an affinity ligand. Other methods for attaching molecules to the surfaces of solid substrate by the use of coupling agents are known in the art, see for example WO98/49557.

Binding of complementary polynucleotide sequences to the immobilised nucleic acid library may be determined by a variety of means such as changes in the optical characteristics of the bound polynucleotide sequence (i.e. by the use of ethidium bromide) or by the use of labelled nucleic acids, such as polypeptides labelled with fluorophores. Other detection techniques that do not require the use of labels include optical techniques such as optoacoustics, reflectometry, ellipsometry and surface plasmon resonance (see WO97/49989).

Thus, the present invention provides a solid substrate having immobilized thereon at least one polynucleotide of the present invention, preferably two or more different polynucleotide sequences of the present invention. In a preferred embodiment the solid substrate further comprises polynucleotide sequences derived from genes other than the BmpB polynucleotide sequence.

Therapeutic Uses

The present invention also can be used as a prophylactic or therapeutic, which may be utilised for the purpose of stimulating humoral and cell mediated responses in swine, thereby providing protection against colonisation with B. hyodysenteriae. Natural infection with B. hyodysenteriae induces good circulating antibody titres against BmpB. Therefore, BmpB amino acid sequence or parts thereof, have the potential to form the basis of a systemically or orally administered prophylactic or therapeutic to provide protection against swine dysentery.

Accordingly, in one embodiment the present invention provides BmpB amino acid sequence or fragments thereof or antibodies that bind said amino acid sequences or the polynucleotide sequences described herein in a therapeutically effective amount admixed with a pharmaceutically acceptable carrier, diluent, or excipient.

The phrase “therapeutically effective amount” is used herein to mean an amount sufficient to reduce by at least about 15%, preferably by at least 50%, more preferably by at least 90%, and most preferably prevent, a clinically significant deficit in the activity, function and response of the animal host. Alternatively, a therapeutically effective amount is sufficient to cause an improvement in a clinically significant condition in the animal host.

The phrase “pharmaceutically acceptable” refers to molecular entities and compositions that are physiologically tolerable and do not typically produce an allergic or similarly untoward reaction, such as gastric upset and the like, when administered to a swine. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carries, particularly, for injectable solutions. Suitable pharmaceutical carriers are described in Martin, Remington's Pharmaceutical Sciences; 18th Ed., Mack Publishing Co., Easton, Pa., (1990).

In a more specific form of the invention there are provided pharmaceutical compositions comprising therapeutically effective amounts of BmpB amino acid sequence or a analogue, fragment or derivative product thereof or antibodies thereto together with pharmaceutically acceptable diluents, preservatives, solubilizes, emulsifiers, adjuvants and/or carriers. Such compositions include diluents of various buffer content (e.g., Tris-HCl, acetate, phosphate), pH and ionic, strength and additives such as detergents and solubilizing agents (e.g., Tween 80, Polysorbate 8C), anti-oxidants (e.g., ascorbic acid, sodium metabisulfite), preservatives (e.g., Thimersol, benzyl alcohol) and bulking substances (e.g., lactose, mannitol). The material may be incorporated into paniculate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Martin, Remington's Pharmaceutical Sciences, 18th Ed. (1990, Mack Publishing Co., Easton, Pa. 18042) pages 1435-1712 that are herein incorporated by reference. The compositions may be prepared in liquid form, or may be in dried powder, such as lyophilised form.

Administration

It will be appreciated that pharmaceutical compositions provided accordingly to the invention may be administered by any means known in the art. Preferably, the pharmaceutical compositions for administration are administered by injection, orally, or by the pulmonary, or nasal route. The BmpB amino acid sequence or antibodies derived there from are more preferably delivered by intravenous, intraarterial, intraperitoneal, intramuscular, or subcutaneous routes of administration. Alternatively, the BmpB amino acid sequence or antibodies derived there from, properly formulated, can be, administered by nasal or oral administration.

Polynucleotide Base Therapy

Also addressed by the present invention is the use of polynucleotide sequences of the invention, as well as antisense and ribozyme polynucleotide sequences hybridisable to a polynucleotide sequence encoding a BmpB amino acid sequence according to the invention, for manufacture of a medicament for modulation of a disease associated B. hyodysenteriae.

Polynucleotide sequences encoding antisense constructs or ribozymes for use in therapeutic methods are desirably administered directly as a naked nucleic acid construct. Uptake of naked nucleic acid constructs by bacterial cells is enhanced by several known transfection techniques, for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Alternatively the antisense construct or ribozymes may be combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular, oral or transdermal administration.

The routes of administration described are intended only as a guide since a skilled practitioner will be able to determine readily the optimum route of administration and any dosage for any particular animal and condition.

Drug Screening Assays

The present invention also provides assays that are suitable for identifying substances that bind to BmpB amino acid sequences. In addition, assays are provided that are suitable for identifying substances that interfere with BmpB amino acid sequences. Assays are also provided that test the effects of candidate substances identified in preliminary in vitro assays on intact cells in whole cell assays.

One type of assay for identifying substances that bind to BmpB amino acid sequences involves contacting a BmpB amino acid sequence, which is immobilised on a solid support, with a non-immobilised candidate substance and determining whether and/or to what extent the BmpB amino acid sequences, and candidate substance bind to each other. Alternatively, the candidate substance may be immobilised and the BmpB amino acid sequence non-immobilised.

In a preferred assay method, the BmpB amino acid sequence is immobilised on beads such as agarose beads. Typically this is achieved by expressing the component as a GST-fusion protein in bacteria, yeast or higher eukaryotic lines and purifying the GST-fusion protein from crude cell extracts using glutathione-agarose beads. The binding of the candidate substance to the immobilised BmpB amino acid sequence is then determined. This type of assay is known in the art as a GST pulldown assay. Again, the candidate substance may be immobilised and the BmpB amino acid sequence non-immobilised.

It is also possible to perform this type of assay using different affinity purification systems for immobilising one of the components, for example Ni-NTA agarose and hexahistidine-tagged components.

Binding of the BmpB amino acid sequence to the candidate substance may be determined by a variety of methods well known in the art. For example, the non-immobilised component may be labelled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Thus, the present invention provides methods of screening for drugs comprising contacting such an agent with a BmpB amino acid sequence or fragment thereof and assaying (i) for the presence of a complex between the agent and the BmpB amino acid sequence or fragment, or (ii) for the presence of a complex between the BmpB amino acid sequence or fragment and a ligand, by methods well known in the art. In such competitive binding assays the BmpB amino acid sequence or fragment is typically labelled. Free BmpB amino acid sequence or fragment is separated from that present in a protein:protein complex, and the amount of free (i.e., uncomplexed) label is a measure of the binding of the agent being tested to the BmpB amino acid sequence or its interference with BmpB amino acid sequence:ligand binding, respectively.

Another technique for drug screening provides high throughput screening for compounds having suitable binding affinity to the BmpB amino acid sequence and is described in detail in. Geysen, PCT published application WO 84/03564, published on Sep. 13, 1984. Briefly stated, large numbers of different small peptide test compounds are synthesised on a solid substrate, such as plastic pins or some other surface. The peptide test compounds are reacted with BmpB amino acid sequence and washed. Bound BmpB amino acid sequence is then detected by methods well known in the art.

This invention also contemplates the use of competitive drug screening assays in which antibodies capable of specifically binding the BmpB amino acid sequence compete with a test compound for binding to the BmpB amino acid sequence or fragments thereof. In this manner, the antibodies can be used to detect the presence of any peptide that shares one or more antigenic determinants of the BmpB amino acid sequence.

Kits of the Invention

The invention also provides kits for screening animals suspected of being infected with B. hyodysenteriae or to confirm that an animal is infected with B. hyodysenteriae, which kit comprises at least a polynucleotide sequence complementary to a portion of the BmpB polynucleotide sequence, packaged in a suitable container, together with instructions for its use.

In a further embodiment of this invention, kits suitable for use by a specialist may be prepared to determine the presence or absence of B. hyodysenteriae in suspected infected swine or to quantitatively measure B. hyodysenteriae infection. In accordance with the testing techniques discussed above, one class of such kits will contain at least the labelled BmpB amino acid sequence or its binding partner, for instance an antibody specific thereto, and directions depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence of B. hyodysenteriae, comprising:
(a) a predetermined amount of at least one labelled immunochemically reactive component obtained by the direct or indirect attachment of the present BmpB amino acid sequence or a specific binding partner thereto, to a detectable label;
(b) other reagents; and
(c) directions for use of said kit.

More specifically, the diagnostic test kit may comprise:
(a) a known amount of the BmpB amino acid sequence as described above (or a binding partner) generally bound to a solid phase to form an immunosorbent, or in the alternative, bound to a suitable tag, or there are a plural of such end products, etc;
(b) if necessary, other reagents, and
(c) directions for use of said test kit.

In a further variation, the test kit may be prepared and used for the purposes stated above, which operates according to a predetermined protocol (e.g. “competitive,” “sandwich,” “double antibody,” etc.), and comprises:
(a) a labelled component which has been obtained by coupling the BmpB amino acid sequence to a detectable label;
(b) one or more additional immunochemical reagents of which at least one reagent is a ligand or an immobilized ligand, which ligand is selected from the group consisting of:
   (i) a ligand capable of binding with the labelled component (a);
   (ii) a ligand capable of binding with a binding partner of the labelled component (a);
   (iii) a ligand capable of binding with at least one of the component(s) to be determined; or
   (iv) a ligand capable of binding with at least one of the binding partners of at least one of the component(s) to be determined; and
(c) directions for the performance of a protocol for the detection and/or determination of one or more components of an immunochemical reaction between the BmpB amino acid sequence and a specific binding partner thereto.

### Examples for Carrying out the Invention

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

Methods of molecular cloning, immunology and protein chemistry, which are riot explicitly described in the following examples, are reported in the literature and are known by those skilled in the art. General texts that described conventional molecular biology, microbiology, and recombinant DNA techniques within the skill of the art, included, for example: Sambrook et al., Molecular Cloning: A Laboratory Manual, Second Edition, Cold Spring Harbor Laboratory Press, Cold Spring Harbor, N.Y. (1989); Glover ed., DNA Cloning: A Practical Approach, Volumes I and II, MRL Press, Ltd., Oxford, U.K. (1985); and Ausubel, F., Brent, R., Kingston, R. E., Moore, D. D., Seidman, J. G., Smith, J. A., Struhl, K. Current protocols in molecular biology. Greene Publishing Associates/Wiley Intersciences, New York (2001).

### EXAMPLES

Oligonucleotide Design

A forward oligonucleotide (BmpB-F13-Xho1) was designed which annealed to the 5′ end of the BmpB polynucleotide sequence. Four reverse oligonucleotides were designed which annealed at approximately 25%, 50%, 75% and 100% of the BmpB polynucleotide sequence. The combination of the forward and reverse oligonucleotides generate amplicons whereby the BmpB polynucleotide sequence would be sequentially truncated by 25% with each reverse oligonucleotide. These oligonucleotides contained terminal endonuclease recognition sequences that would allow cloning of the polymerase chain reaction (PCR) product into the multiple cloning site (MCS) of the pTrcHis vector. The pTrcHis-F oligonucleotide annealed upstream from the pTrcHis MCS and was used for reading frame analysis of the recombinant plasmids. Oligonucleotide sequences are shown below.

| | |
|---|---|
| BmpB-F13-Xho1 | |
| 5′ AAACTCGAGTTATTATTGGTATCATCAGC 3′ | (SEQ ID NO:18) |
| BmpB-R195-ECoR1 | |
| 5′ TATGAATTCATCAGAGAAAGATACTAGCTC 3′ | (SEQ ID NO:19) |
| BmpB-R411-EcoR1 | |
| 5′ TCCGAATTCAGAAGGGTCATTAGGTATAGC 3′ | (SEQ ID NO:20) |
| BmpB-R613-EcoR1 | |
| 5′ GATGAATTCCGAAGTATATAGCATAGTTTC 3′ | (SEQ ID NO: 21) |
| BmpB-R809-EcoR1 | |
| 5′ TATGAATTCCAAGTAGGAAGATAAGAACC 3′ | (SEQ ID NO:22) |
| pTrcHis-F | |
| 5′ CAATTTATCAGACAATCTGTGTG 3′ | (SEQ ID NO:23) |

PCR Conditions

The PCR reaction (100 μl) consisted of 20 mM Tris-HCl (pH 8.8), 2 mM MgSO₄, 10 mM KCl, 10 mM (NH₄)SO₄, 0.1% (v/v) Triton X-100, 100 μg/ml BSA, 2 mM of each dNTP, 50 pmol of each oligonucleotide, 4 U of Pfu DNA polymerase (Promega) and 2 ng of B. hyodysenteriae high molecular weight DNA. The amplification consisted of an initial denaturation at 95° C. for 2 min followed by 30 cycles of 95° C. for 30 s, 60° C. for 30 s, 72° C. for 1 min, and followed by an indefinite hold at 14° C.

Oligonucleotide combinations of BmpB-F13-Xho1 with BmpB-R195-EcoR1 generated a 182 bp insert, BmpB-F13-Xho1 with BmpB-R411-EcoR1 generated a 398 bp insert, BmpB-F13-Xho1 th BmpB-R613-EcoR1 generated a 601 bp insert, and BmpB-F13-Xho1 with BmpB-R809-EcoR1 generated a 796 bp insert.

Cloning of pTrcHis

The PCR products were purified using the BresaSpin PCR Purification Columns (GeneWorks) according to the manufacturer's instructions. The pTrcHis vector and the purified PCR products were digested with 5 U of Xho1 and 5 U of EcoR1 in 100 mM-Tris-HCl (pH 7.5), 50 mM NaCl, 10 mM MgCl₂, 1 mM dithiothreitol, 0.025% (v/v) Triton X-100, and 100 μg/ml BSA at 37° C. overnight. Digested pTrcHis and PCR products were purified using the BresaSpin PCR Purification Columns according to the manufacturer's instructions. Ligation of the pTrcHis vector and BmpB inserts occurred at 14° C. overnight with a 1:1 molar ratio. The ligation reaction consisted of 30 mM Tris-HCl (pH 7.8), 10 mM MgCl₂, 10 mM dithiothreitol. 1 mM ATP, and I U T4 DNA ligase (Promega). Ligation products were transformed into chemically competent Escherichia coli JM109 cells using the heat-shock method, and plated onto LB agar plates containing 100 μg/ml ampicillin.

Sequencing of Recombinant Plasmids

Colonies, which survived ampicillin selection, were grown in LB broth culture and their plasmids extracted using the Qiagen Plasmid Mini-prep Columns (Qiagen) according to the manufacturers instructions. Plasmids were sequenced with the pTrcHis-F oligonucleotide using the Taq DyeDeoxy™ Terminator Cycle Sequencing Kit supplied by Applied Biosystems. The sequences were viewed and the reading frame aligned using the SeqEd and DNA Strider programs.

Expression of Recombinant Plasmids

Plasmids were transformed into chemically competent E. coli BL21 cells using the heat-shock method and plated onto LB-ampicillin agar plates. Colonies, which survived the ampicillin selection, were re-streaked onto fresh LB-ampicillin agar plates before inoculation into 10 ml LB-ampicillin broth for overnight culture at 37° C. One ml of overnight culture was added to 50 ml LB-ampicillin broth and incubated at 37° C. with vigorous shaking. After 3 h incubation, the cultures were induced with 0.5 mM IPTG and the cells returned to 37° C. for a further 3 h.

Purification of Truncated Fusion Protein

Cells were immediately harvested by centrifugation at 1,500 g for 10 min. The histidine fusion proteins were purified from the cell pellet under denaturing conditions using the Qiagen Ni-NTA Spin Kit (Qiagen) according to the manufacturer's instructions.

SDS-PAGE and Western Blotting of Purified Fusion Proteins

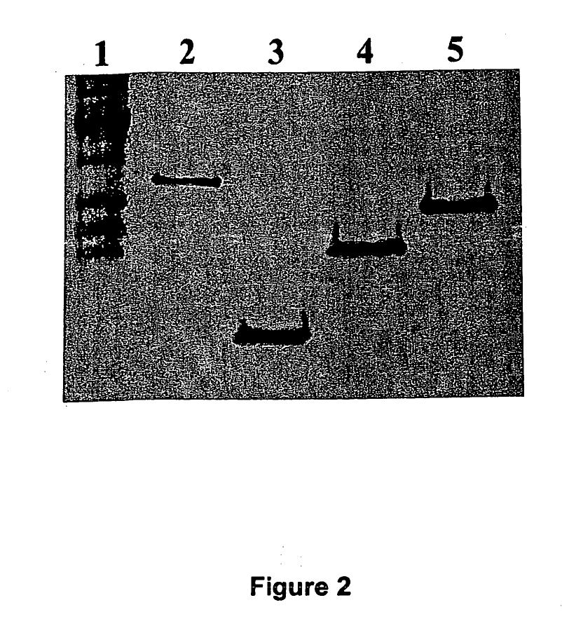

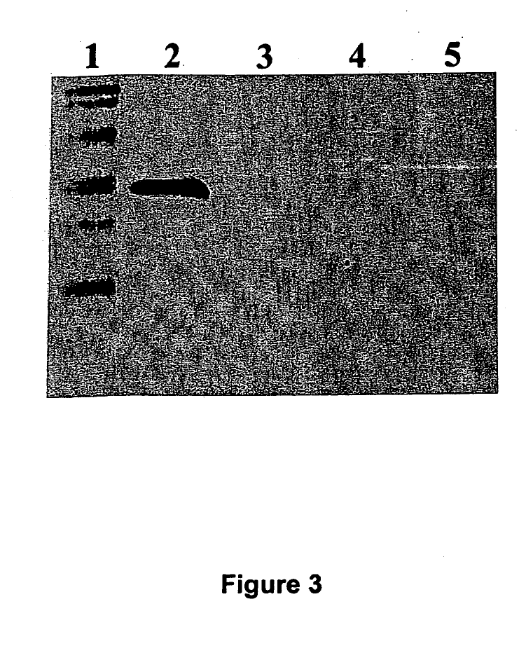

Thirty μl of purified fusion protein was added to 10 μl of Tricine sample buffer and boiled for 5 min. Ten μl of the boiled sample was loaded onto a 12% (w/v) SDS-PAGE gel and electrophoresed for 2 h at 150V using the Tricine buffer system (Schagger and von Jagow, 1987). The separated proteins were electro-transferred to nitrocellulose membrane at 100V for 1 h. The membrane was blocked with TBS-skim milk (5% w/v) for 1 h at room temperature (RT) followed by incubation with monoclonal antibody BJUSH1 for 1 h. Binding of BJUSH1 was detected using goat anti-mouse IgG (H+L) alkaline phosphatase for 1 h at RT. The membrane was developed using the Biorad Alkaline Phosphatase Development Kit (Biorad) according to the manufacturer's instructions. To confirm expression of the fusion proteins, a second membrane was blotted using a monoclonal antibody directed against the hexa-histidine fusion (Anti-His). Western blot analysis using the Anti-His antibody showed that all truncated BmpB proteins were expressed and purified (FIG. 2). Western blot analysis using the monoclonal antibody BJL/SH1 showed that only the full recombinant BmpB protein was reactive with BJL/SH1 and the truncated recombinant BmpP proteins, did not react (FIG. 3). This indicated the location of the BJL/SHI epitope to be in the 613-809 bp region of the BmpB polynucleotide sequence (i.e. C-terminal end of BmpB amino acid sequence).

Cloning, Expression and Purification of BmpB-F604/R809 Portion

The cloning, expression and purification of the BmpB-F604/R809 C-terminal portion of BmpB: polynucleotide sequence was perform as described above. Oligonucleotides used to generate the BmpB-F604/R809 insert are shown below.

| | |
|---|---|
| BmpB-F604-Xho1 | |
| 5′ AACCTCGAGATATACTTCGGTTTGAATCCT | (SEQ ID NO:24) |
| G 3′ | |
| BmpB-R809-ECoR1 | |
| 5′ TATGAATTCCAAGTAGGAAGATAAGAACC 3′ | (SEQ ID NO:25) |

BmpB-F604/R809 ELISA Conditions

Purified BmpB-F604/R809 was diluted in bicarbonate/carbonate coating buffer (pH 9.6) to a working dilution of 3 μg/ml. One hundred μL of the working dilution was used to coat each well of the 96 well microtitration plate. Coating was allowed to occur at 4° C. overnight. The wells were blocked with 150 μl of PBS-skim milk powder (5% w/v) for 1 h at RT. The plate was washed three times with PBST (0.1% v/v) before applying the pig serum. Sera were diluted 1:100 with PBST and 100 μl incubated in the wells with gentle mixing for 2 h at RT. After washing the plate five times with 150 μl of PBST, 100 μL of diluted (1:2000) goat anti-pig IgG HRP was added to each well. The plates were incubated for 1 h at RT with gentle mixing, before washing the plate five times with PBST. To remove the residual Tween 20, the plate was washed an additional three times with 150 μl of PBS. One hundred μl of K-Blue TMB Substrate Solution (ELISA Systems) was added to each well and incubated for 20 min at RT to allow colour development, before reading the optical density (OD) at 655 nm.

BmpB-F604 ELISA Test

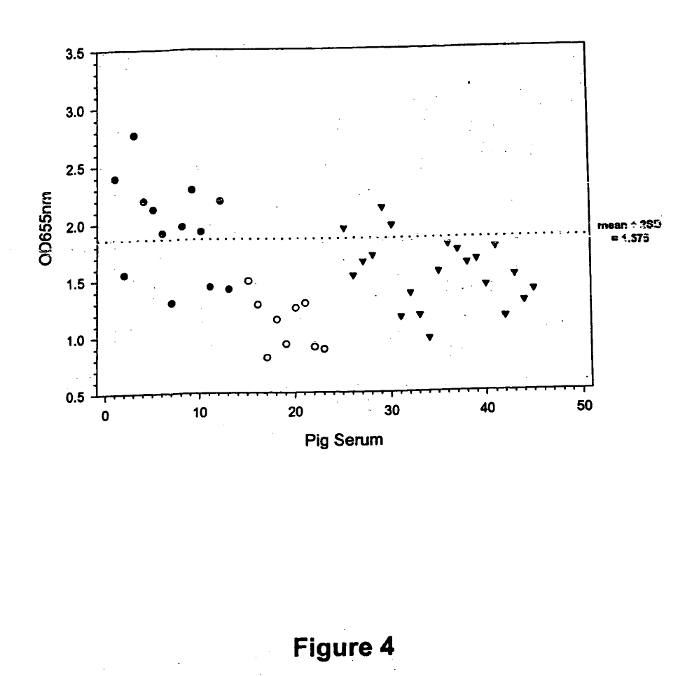

Serum front pigs naturally infected with B. hyodysenteriae, healthy pigs, and pigs from a farm experiencing a swine dysentery outbreak were analysed using the BmpB-F604/R809 ELISA (FIG. 4). The ELISA test was able to distinguish the naturally infected farm from the healthy farm.

Analysis of BmpB in Brachyspira Species

Polymerase Chain Reaction (PCR)

Two oligonucleotides which annealed to the 3′-OH and 5′-OH ends of the BmpB polynucleotide sequence were designed and optimised for PCR detection of the BmpB polynucleotide from 82 Brachyspiral genomic DNA: 48 strains of Brachyspira hyodysenteriae, 18 strains of Brachyspira pilosicoli, 12 strains of Brachyspira intermedia, 8 strains of Brachyspira murdochii, 4 strains of Brachyspira innocens, 2 strains of “Brachyspira canis”, 1 strain of Brachyspira alvinipulli and 1 strain of Brachyspira aalborgi.

The oligonucleotides used were BmpB-L1 (5′-AGGGATGAGGATAACAGTC-3′) (SEQ NO:26) and BmpB-R2 (5′-ATGAGTACAGGTAAAGATGC-3′) (SEQ ID NO:27) which anneal to complementary sequences flanking the BmpB polynucleotide sequence. The gene was amplified by PCR in a 50 μl total volume using Taq DNA polymerase (Biotech International) and Pfu DNA polymerase (Promega). The amplification mixture consisted of 1×PCR buffer (containing 1.5 mM of MgCl₂), 0.5 U of Taq DNA polymerase, 0.05 U Pfu DNA polymerase, 0.2 μM of each dNTP (Amersham Pharmacia Biotech), 0.5 FM of the oligonucleotide pair (BmpB-L1, BmpB-R2), and 2.5 μl chromosomal template DNA. Chromosomal DNA was prepared previously using the DNeasy Tissue Kit (Qiagen) according to the manufacturer's instructions. Cycling conditions involved an initial template denaturation step of 5 min at 94° C., followed by 30 cycles of denaturation at 94° C. for 30 sec, annealing at 55° C. for 15 sec, and oligonucleotide extension at 68° C. for 2 min. The PCR products were subjected to electrophoresis in 1.5% (w/v) agarose gels in 1×TAE buffer (40 mM Tris-acetate, 1 mM EDTA), stained with a 1 μg/ml ethidium bromide solution and viewed over UV light.

Sequencing of the BmpB Polynucleotide Sequence Present in other Brachyspira spp.

PCR products from the Brachyspira spp. were purified using the UltraClean PCR Clean-up Kit (Mo Bio Laboratories), according to the manufacturer's instructions. Sequencing of the PCR product was performed using the BmpB-L1 and BmpB-R2 primers. Each sequencing reaction was performed in a 10 μl volume consisting of PCR product (50 ng), primer (2 pmol), and ABI PRISM™ Dye Terminator Cycle Sequencing Ready Reaction Mix (4 μl) (PE Applied Biosystems); Cycling conditions involved a 2 minute denaturing step at 96° C., followed by 25 cycles of denaturation at 96° C. for 10 seconds, oligonucleotide annealing at 55° C. for 5 seconds, and oligonucleotide extension at 60° C. for 4 minutes.

Residual dye terminators were removed from the sequencing products by precipitation with 95% (v/v)-ethanol containing 120 mM sodium acetate (pH 4.6), and vacuum dried. The sequencing products were analysed using an ABI 373A DNA Sequencer. Sequence results were edited, compiled and compared using SeqEd v1,0,3 and Vector NTI version 6.

Results

The BmpB polynucleotide sequence was found to be present in all strains of B. hyodysenteriae and all strains of B. innocens tested, but was not present in any strains of B. pilosicoli, B. intermedia, B. murdochii, “B. canis”, B. alvinipulli or B. aalborgi. Eight strains of B. hyodysenteriae and all four strains of B. innocens were selected for sequencing of the BmpB polynucleotide present. Table 2 and 3 summarises the level of homology between the BmpB polynucleotide sequence of the B. hyodysenteriae strains and B. innocens strains compared to the originally sequenced BrmpB polynucleotide sequence of B. hyodysenteriae P18A. The BmpB polynucleotide sequence of the eight B. hyodysenteriae strains showed 98.5-99.8% homology with the BmpB polynucleotide sequence of B. hyodysenteriae P18A (Table 2). The BmpB amino acid sequence of the eight B. hyodysenteriae strains showed 98.5-99.3% homology with the BmpB amino acid sequence of B. hyodysenteriae P18A (Table 3). All Western Australian isolates shared the same BmpB amino acid sequence homology with strain P18A, although the sequence from these isolates was not identical. The BmpB polynucleotide sequence of B. innocens strains showed slightly higher variation with between 96.1-99.1% homology with the BmpB polynucleotide sequence of B. hyodysenteriae P18A. The BmpB amino acid sequence of B. innocens strains showed between 97.499.3% homology with the BmpB amino acid sequence of B. hyodysenteriae P18A. The high level of homology between the different strains of B. hyodysenteriae and B. innocens suggests that the BmpB polynucleotide sequence is highly conserved within these species.

The polynucleotide sequence homology of the originally sequenced BmpB of B. hyodysenteriae P18A with BmpB of other B. hyodysenteriae and B. innocens strains is shown in Table 2 below. All strains possess an 816 base pair (bp) polynucleotide.

TABLE 2

| Homology of | | | | | |
|---|---|---|---|---|---|
| B. hyodysenteriae strains | | | Homology of B. innocens strains | | |
| | Identity | | | | Homology |
| Strain | (bp) | Homology (%) | Strain | Identity (bp) | (%) |
| B78^{T} | 810 | 99.3 | B256^{T} | 809 | 99.1 |
| B169 | 812 | 99.5 | 4/71 | 809 | 99.1 |
| B204 | 814 | 99.8 | Q91 | 784 | 96.1 |
| BW1 | 813 | 99.6 | West A | 784 | 96.1 |
| WA4 | 813 | 99.6 | | | |
| WA5 | 804 | 98.5 | | | |
| WA6 | 804 | 98.5 | | | |
| WA15 | 813 | 99.6 | | | |
| WA16 | 813 | 99.6 | | | |

Amino acid (aa) sequence homology of the originally sequenced BmpB lipoprotein of B. hyodysenteriae P18A with BmpB lipoprotein of other B. hyodysenteriae and B. innocens strains are shown in Table 3 below. All strains posses a 271 amino acid pro-lipoprotein.

TABLE 3

| Homology (%) of | | | Homology (%) of | | |
|---|---|---|---|---|---|
| B. hyodysenteriae strains | | | B. innocens strains | | |
| | Identity | | | | Homology |
| Strain | (aa) | Homology (%) | Strain | Identity (aa) | (%) |
| B78^{T} | 269 | 99.3 | B256^{T} | 269 | 99.3 |
| B169 | 267 | 98.5 | 4/71 | 269 | 99.3 |
| B204 | 269 | 99.3 | Q91 | 264 | 97.4 |
| BW1 | 269 | 99.3 | West A | 264 | 97.4 |
| WA4 | 268 | 98.9 | | | |
| WA5 | 268 | 98.9 | | | |
| WA6 | 268 | 98.9 | | | |
| WA15 | 268 | 98.9 | | | |
| WA16 | 268 | 98.9 | | | |

Evaluation of Immunisation for Protection against Brachyspira hyodysenteriae Colonisation in Pigs.

Animals

Thirty female weaner pigs (Large White×Landrace×Duroc) weaned at 21 days of age were purchased at weaning from a commercial piggery. The pigs were weighed and ear-tagged, then randomly assigned to three groups of ten, each group housed in an adjacent pen (open wire-mesh partitions) in one room of an isolation animal house. Pigs were fed ad libidum on a commercial pelleted weaner diet that did not contain antibiotics. The three groups included:
i) Group A: received no vaccination;
ii) Group. B: received 1 mg protein with adjuvant intramuscularly, followed 3 weeks later by 1 mg protein in solution via stomach tube (im/oral).
iii) Group C: received 1 mg protein with adjuvant intramuscularly, followed 3 weeks later by another 1 mg protein with adjuvant intramuscularly (im/im).
   Immunisation and Infection Protocols

One day after arrival, pigs in groups B and C received their first vaccination. These pigs were immunised intramuscularly (im) in the neck with 1 mg of recombinant BmpB lipoprotein of B. hyodysenteriae emulsified in Freund's incomplete adjuvant to a volume of 2 ml. Three weeks later, pigs in group B were given an oral boost with 1 mg recombinant BmpB in 10 ml phosphate buffered saline (PBS) administered by stomach tube, whilst pigs in group C received a second intramuscular vaccination identical to the first vaccination. Two weeks later pigs in all three groups were challenged with 50 ml of exponential log-phase (˜10⁸/ml) Australian B. hyodysenteriae strain “Brentwood/Q02”, using a stomach tube. Challenge was repeated over five consecutive days.

Blood samples were collected from the jugular vein prior to the first vaccination, just prior to the second vaccination, prior to the first day of challenge, and at post-mortem. Sera were collected using standard procedures and tested by ELISA for antibodies to the vaccine antigen as well as to a whole-cell preparation of the bacterial strain used in the challenge.

Following challenge, all pigs were swabbed rectally three times per week, and the swabs cultured anaerobically on selective agar. Faeces was observed for signs of diarrhoea containing blood and/or mucus, and obvious signs of weight loss in the animal (SD). Observation of normal solid faeces without signs of diarrhoea containing blood and/or mucus, and no obvious signs of weight loss in the animal indicated no clinical signs of SD. Within 24 hours of observing diarrhoea typical of SD, pigs were removed for post-mortem. The remaining pigs which did riot develop clinical signs of SD were removed for post-mortem at the end of the experimental period. The post mortems at the end of the experiment were carried out over a three day period, between 20 and 23 days after the last day of the experimental inoculation.

Samples of colonic epithelia were collected at post-mortem and tested for specific immunoglobulin content by ELISA. The caecae from all pigs were swabbed and cultured for B. hyodysenteriae in the same manner as for faeces.

Spirochaetal Culture

Swabs taken from faeces and caecums were streaked onto Trypticase Soy agar plates containing defibrinated sheep blood (5% v/v), spectinomycin (400 μg/ml), colistin (25 μg/ml) and vancomycin (25 μg/ml). Plates were incubated at 37° C. in an aerobic environment for seven days. Spirochaetes were identified as B. hyodysenteriae on the basis of strong beta-haemolysis and microscopic morphology. A subset of isolates were sub-cultured and confirmed as B. hyodysenteriae using a species-specific PCR.

ELISA (Serum)

The weils of micro-titre plates (Immulon 4HBX, Dynex) were coated with either (i) purified BmpB (500 ng/ml) (100 μl), or (ii) sonicated and cleared B. hyodysenteriae whole-cells (1 μg/ml) in carbonate buffer (pH 9.6) (100 μl). The plates were incubated overnight at 4° C.

A blocking solution (150 μl) of PBS-skim milk (5% w/v) was added to the wells of the plates and the plates incubated for 1 hour at room temperature, with mixing and then washed three times with 150 μl of PBST (0.05% vv).

Pig sera was diluted 200-fold in 100 μl of PBST-skim milk (0.5% w/v), added to the wells of the plates and incubated at room temperature for 2 hours, with mixing. Plates were then washed, as outlined above, and 100 μl of goat anti-swine IgG (Whole molecule)-HRP diluted 5000-fold in PBST-skim milk (0.5% w/v) was added to each well and plates incubated for 1 hour at room temperature. The plates were then washed as above and TMB substrate (100 μl) was added to each well.

Colour development at room temperature was stopped after 10 minutes by the addition of 1M sulfuric acid (50 μl). The optical density of each well was read at 450 nm using a micro-plate reader (Biorad Model 3550-UV).

ELISA (Mucosal)

Mucosal antibodies were extracted from a 5 cm×5 cm section of the proximal colon. The epithelium was briefly rinsed to remove digesta, then stripped off with a scalpel blade and the epithelial cells were resuspended in 4 ml of PBS containing 1% (w/v) BSA, 2 mM PMSF, 1 mM EDTA and 0.2% (w/v) sodium azide. Suspensions were mixed vigorously for 1 minute and centrifuged at 14,000 rpm for 10 minutes. The supernatant was removed and an aliquot of the sample (100 μl) used for ELISA. ELISA was performed as for the serum ELISA discussed, above.

Results and Discussion

Serological Response to the Vaccination

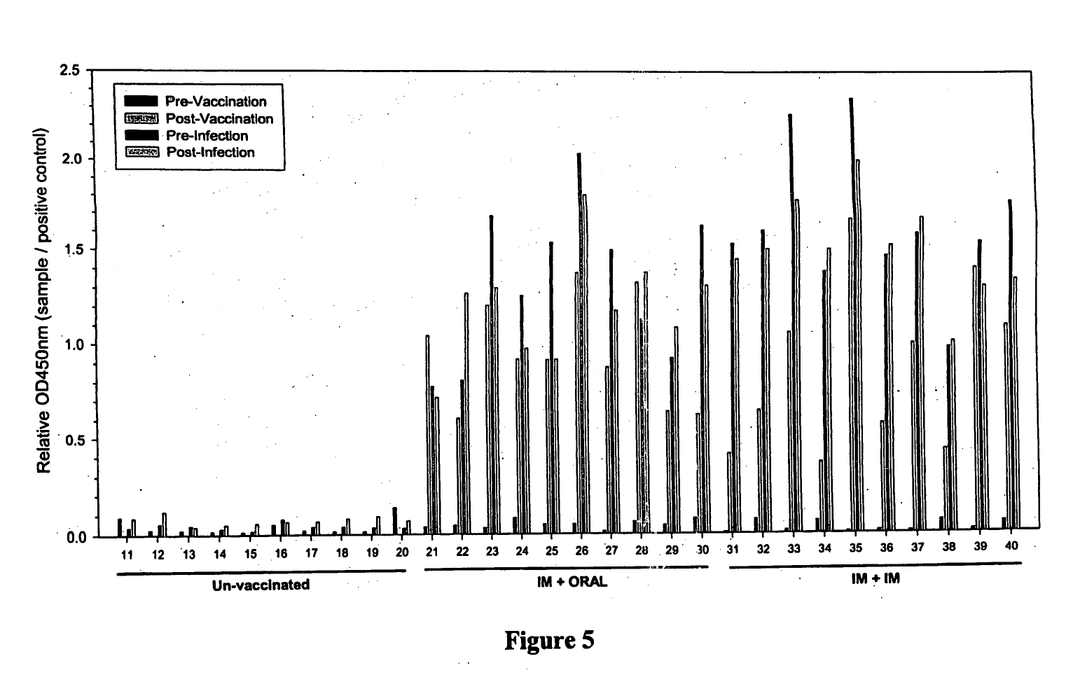

The systemic immune response of the pigs to the recombinant vaccine is shown in FIG. 5. Unvaccinated control pigs (group A) did not have circulating antibody to the BmpB lipoprotein and no antibody developed after experimental infection.

Vaccinated pigs developed good primary and secondary response to the vaccination, with the exception of two pigs (21 and 28) in group B which received the boost orally.

Most pigs did not show a boost to circulating antibody after experimental infection, although pigs 22, 28 and 29 from the oral vaccination group (group B) did show a moderate boost in circulating antibody response following challenge. None of the unvaccinated control pigs (group A) showed an antibody response following oral challenge.

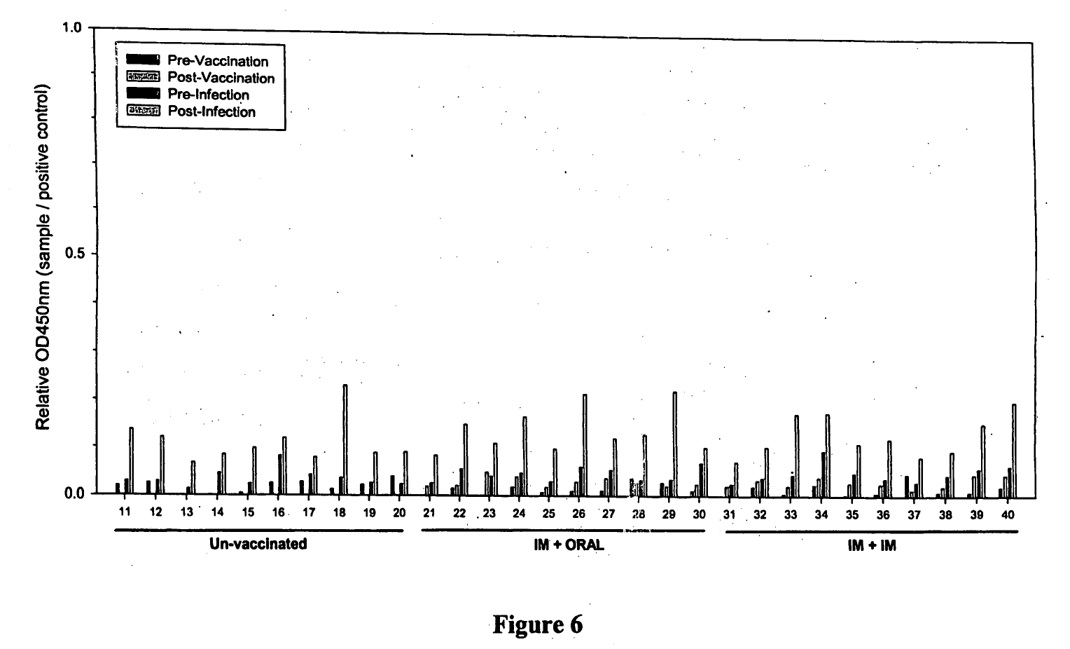

FIG. 6 shows the results of the ELISA experiment on pig sera from all three groups for the systemic antibody response of the pigs to a whole-cell preparation of the B. hyodysenteriae strain used for the challenge. All pigs showed an increase in antibody levels following challenge. However, these levels were lower than the levels seen against recombinant BmpB. In addition, Western blot 15, analysis of pooled pig serum (diluted 1:50) against the same whole-cell preparation failed to detect any visible reactivity, thus confirming the low antibody titres present (data not shown).

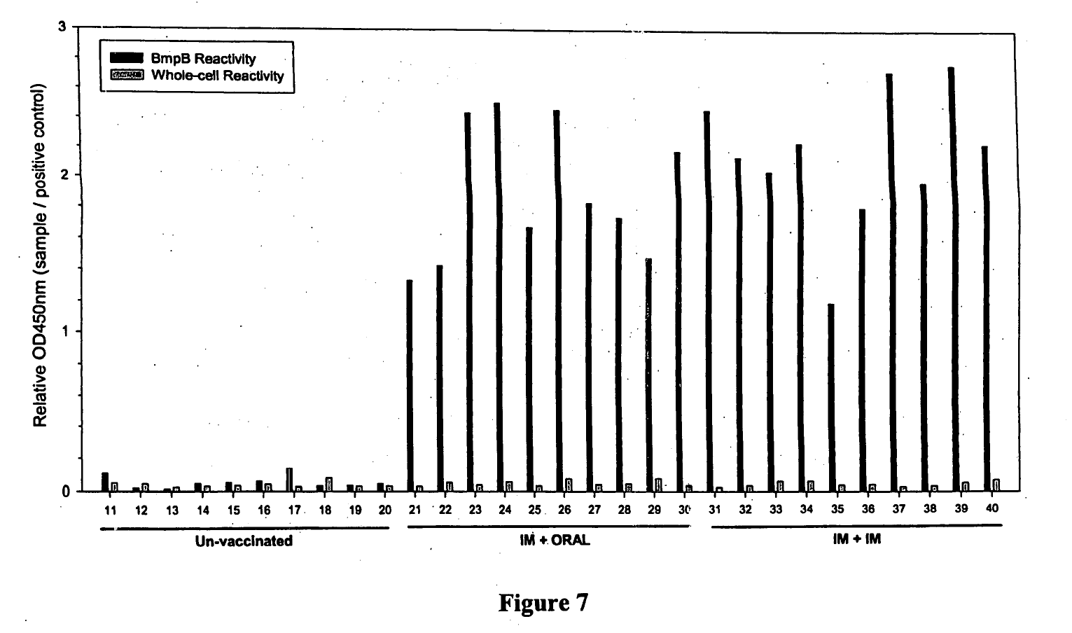

The mucosal antibody response of the pigs to the vaccination and challenge (samples collected post-mortem) is shown in FIG. 7. The control pigs did not show any local responses to either the recombinant BmpB or the whole-cell preparation, despite being infected. All vaccinated pigs showed a moderate to high local antibody response to the vaccination, thus indicating the presence of potentially protective antibody at the site of colonisation. It is unknown whether this local response was due to the vaccination alone or was boosted by challenge. However, all pigs failed to show a local response to the whole-cell preparation despite being infected with B. hyodysenteriae, thus it is probable that the local response was a result of vaccination.

Excretion of Brachyspira hyodysenteriae in the Faeces

The pattern of faecal excretion of B. hyodysenteriae detected in pigs from the three groups is shown in Tables 4-6 respectively. The tables present data from individual facecal culture for unvaccinated pigs (Group A), vaccinated pigs (Group B) and vaccinated pigs (Group C) after oral challenge with B. hyodysentenae. Pigs were removed for post-mortem when diarrhoea was observed, or else between day 20 and day 23. The (−) symbol represents culture negative, (+) represents culture positive and (↓) indicates that no culture result was available as the pig had been removed for post mortem.

Table 4 shows the individual faecal culture results for the unvaccinated pigs (Group A) after oral challenge with B. hyodysenteriae. The. day represents the number of days post infection. For the unvaccinated control pigs, excretion of B. hyodysenteriae was first detected in two pigs (11 and 18) six days after the end of experimental infection. One pig (14) was killed before the end of the experiment (it had diarrhoea, but subsequently was found not to have SD). Of the remaining nine pigs, eight were found to be colonised by B. hyodysenteriae—on the basis of having positive faecal cultures. The appearance of clinical signs of SD was always preceded by the presence of positive faecal cultures.

TABLE 4

| Pig | Day | Day | Day | Day | Day | Day | Day | Day | Day | Day |
|---|---|---|---|---|---|---|---|---|---|---|
| Number | −9 | 3 | 6 | 8 | 10 | 14 | 16 | 20 | 22 | 23 |
| 11 | − | − | + | + | + | ↓ | ↓ | ↓ | ↓ | ↓ |
| 12 | − | − | − | − | − | − | − | − | − | + |
| 13 | − | − | − | + | + | + | + | ↓ | ↓ | ↓ |
| 14 | − | − | − | − | − | − | − | ↓ | ↓ | ↓ |
| 15 | − | − | − | − | − | − | + | + | + | ↓ |
| 16 | − | − | − | − | + | ↓ | ↓ | ↓ | ↓ | ↓ |
| 17 | − | − | − | − | − | − | + | + | ↓ | ↓ |
| 18 | − | − | + | + | + | ↓ | ↓ | ↓ | ↓ | ↓ |
| 19 | − | − | − | − | − | + | + | ↓ | ↓ | ↓ |
| 20 | − | − | − | − | − | − | − | − | − | − |
| % culture | 0% | 0% | 20% | 30% | 40% | 29% | 57% | 50% | 33% | 50% |
| positive | (0/10) | (0/10) | (2/10) | (3/10) | (4/10) | (2/7) | (4/7) | (2/4) | (1/3) | (1/2) |

Results for pigs vaccinated intramuscularly then orally (Group B) are shown in Table 5. The first faecal positive pig (pig 28) was detected fourteen days after experimental infection. One pig was removed due to lameness. Of the remaining nine pigs, five were faecal positive at some point, although one of these five was subsequently culture negative at post-mortem.

TABLE 5

| Pig | Day | Day | Day | Day | Day | Day | Day | Day | Day | Day |
|---|---|---|---|---|---|---|---|---|---|---|
| Number | −9 | 3 | 6 | 8 | 10 | 14 | 16 | 20 | 22 | 23 |
| 21 | − | − | − | − | − | − | − | − | + | ↓ |
| 22 | − | − | − | − | − | − | − | − | − | − |
| 23 | − | − | − | − | − | − | − | − | + | − |
| 24 | − | − | − | − | − | − | − | − | − | ↓ |
| 25 | − | − | − | − | − | − | − | − | − | ↓ |
| 26 | − | − | − | − | − | − | − | − | − | ↓ |
| 27 | − | − | − | − | − | − | − | + | + | ↓ |
| 28 | − | − | − | − | − | + | + | + | ↓ | ↓ |
| 29 | − | − | − | − | − | − | − | + | + | ↓ |
| 30 | − | − | − | − | − | ↓ | ↓ | ↓ | ↓ | ↓ |
| (lame) | | | | | | | | | | |
| % culture | 0% | 0% | 0% | 0% | 0% | 11% | 11% | 33% | 50% | 0% |
| positive | (0/10) | (0/10) | (0/10) | (0/10) | (0/10) | (1/9) | (1/9) | (3/9) | (4/8) | (0/2) |

For the group of pigs vaccinated twice intramuscularly (Group C; Table 6), the first pig became culture positive after six days (pig 31). Overall, seven of the ten pigs were faecal culture positive at some time point, although not all went on to develop dysentery.

TABLE 6

| Pig | Day | Day | Day | Day | Day | Day | Day | Day | Day | Day |
|---|---|---|---|---|---|---|---|---|---|---|
| Number | −9 | 3 | 6 | 8 | 10 | 14 | 16 | 20 | 22 | 23 |
| 31 | − | − | + | + | + | ↓ | ↓ | ↓ | ↓ | ↓ |
| 32 | − | − | − | − | − | − | − | − | − | − |
| 33 | − | − | − | − | − | − | − | − | − | − |
| 34 | − | − | − | − | − | − | − | + | ↓ | ↓ |
| 35 | − | − | − | − | − | − | − | + | ↓ | ↓ |
| 36 | − | − | − | − | − | − | − | − | ↓ | ↓ |
| 37 | − | − | − | − | + | + | + | + | ↓ | ↓ |
| 38 | − | − | − | − | − | − | − | − | − | ↓ |
| 39 | − | − | − | − | − | − | − | + | + | ↓ |
| 40 | − | − | − | − | − | + | − | + | ↓ | ↓ |
| % | 0% | 0% | 10% | 10% | 20% | 22% | 11% | 56% | 25% | 50% |
| Culture | (0/10) | (0/10) | (1/10) | (1/10) | (2/10) | (2/9) | (1/9) | (5/9) | (1/4) | (1/2) |
| Positive | | | | | | | | | | |

Development of Disease and Lesions at Postmortem

Of the 10 unvaccinated pigs, seven developed clinical signs of dysentery and had lesions of severe mucohaemorrhagic colitis at postmortem (Table 7). One pig (14) was removed early because it had diarrhoea, but it was culture negative and has no signs of colitis. The other two pigs were healthy at slaughter, but one was culture positive (12).

TABLE 7

| | | | Severity of colonic | |
|---|---|---|---|---|
| Pig | PM Culture | Clinical SD | lesions | Reason for PM |
| 11 | + | + | severe | diarrhoea |
| 12 | + | − | — | EOE |
| 13 | + | + | severe | diarrhoea |
| 14 | − | − | — | SD suspect |
| 15 | + | + | severe | diarrhoea |
| 16 | + | + | severe | diarrhoea |
| 17 | + | + | severe | diarrhoea |
| 18 | + | + | severe | diarrhoea |
| 19 | + | + | severe | diarrhoea |
| 20 | − | − | — | EOE |
| 21 | + | + | severe | diarrhoea |
| 22 | − | − | — | EOE |
| 23 | − | − | mild | EOE |
| 24 | − | − | — | EOE |
| 25 | − | − | — | EOE |
| 26 | − | − | — | EOE |
| 27 | + | − | mild | EOE |
| 28 | + | + | mild | diarrhoea |
| 29 | + | + | severe | diarrhoea |
| 30 | − | − | — | lame pig |
| 31 | + | + | mild | diarrhoea |
| 32 | − | − | — | EOE |
| 33 | + | − | — | EOE |
| 34 | + | + | mild | diarrhoea |
| 35 | + | − | — | EOE |
| 36 | − | − | — | EOE |
| 37 | + | + | mild | diarrhoea |
| 38 | − | − | — | EOE |
| 39 | + | + | severe | diarrhoea |
| 40 | + | − | — | EOE |

In comparison, three pigs in group B, (vaccinated intramuscularly then orally) developed diarrhoea. Two of these had severe lesions of mucohaemorrhagic colitis at postmortem whilst the third only had mild localised lesions. One pig was removed because of lameness, and had no lesions. The remaining five pigs stayed healthy and survived to the end of the experiment without developing diarrhoea. Two of these healthy pigs had mild lesions limited to the proximal colon at postmortem. Of these two pigs, Pig 23 was culture negative at postmortem, but had delivered a positive faecal culture the daV before. Pig 27 was culture positive at postmortem, and had been faecal positive for several days before slaughter.

Further, four of the Group C pigs (vaccinated twice intramuscularly) developed diarrhoea, of which all four were culture positive at slaughter. Only one of the four pigs had severe lesions in the colon, with the other three having only mild and/or localised lesions. Of the remaining six pigs in group C, three were culture positive at postmortem, and all three were also faecal culture positive prior to slaughter. None of these six pigs had colonic lesions at-slaughter.

### CONCLUSION

This study successfully reproduced swine dysentery, with seven of ten control pigs developing disease one uninfected pig was removed early, and may have gone on to develop disease). Faecal excretion was first detected six days after the start of experimental infection, thus emphasising that the system of challenge was effective. All pigs that developed diarrhoea had severe and extensive lesions in their large intestines at postmortem. Furthermore, these clinically affected animals all tended to excrete spirochaetes in their faeces on between two to four sampling times before they developed disease. This is consistent with there being a slow build up of spirochaete numbers in the large intestine, and progressive development of lesions along the colon to a point where diarrhoea and dysentery developed.

Both vaccination regimens (Group B and C) provided a degree of protection against both colonisation and disease, although neither gave complete protection. There were less total days of colonisation, and colonisation tended to occur later with both vaccinated groups than with the controls, but especially with the intramuscular/oral group (B). There was also less diarrhoea (3/9 pigs and 4/10 pigs) in vaccine groups B and C respectively, and fewer animals with severe lesions in the colon at postmortem (2/9 and 1/10 for groups B and C respectively).

The remaining pigs with diarrhoea in the two groups had only localised and mild colonic lesions. Two pigs in group B had mild lesions in the proximal colon, but were robust and clinically healthy. Whether some or all these pigs with mild colonic lesions and/or recent colonisation in the absence of lesions would have gone on to develop more severe lesions and/or more severe clinical signs is not known. Given that they tended to become colonised later than the control group, this possibility cannot be discounted. In future experiments it would be useful to keep vaccinated animals for longer after experimental infection to determine whether disease ultimately would occur.

Given the fact that the vaccinated pigs tended to become infected later than the control pigs, and that all were housed in the same room, it is possible that the vaccinated pigs received additional challenge from the diseased control pigs (group A). In a commercial piggery it is likely that all susceptible pigs would be vaccinated, and hence the infectious load would be reduced. In future experiments it would be useful to house the infected control pigs and the vaccinated pigs in different rooms to reduce exposure of the vaccinated pigs to an artificially high re-challenge from control pigs with SD.

Whilst the vaccines both induced systemic and colonic antibody production against the BmpB lipoprotein, there was no clear correlation between these titres and protection/disease. Experimental infection alone also did not induce titres against the BmpB lipoprotein. It is possible that the specific protection that occurred following vaccination was related to IgA titres in the colon, and/or to cell mediated responses in the colon, but neither of these possibilities were explored. This would form a useful component of future studies on the vaccine.

Overall, the study provided encouraging results that suggested that BmpB has potential as a protective vaccine component for use in the control of SD.

Further Evaluation of Immunisation for Protection against Brachyspira Hyodysenteriae Colonisation in Pigs

A pig vaccination trial for swine dysentery (SD) was undertaken using recombinant BmpB liporotein as the vaccine candidate to determine whether the results described above could be repeated. In addition, the suitability of VSA3 as an adjuvant for the vaccine was also investigated. Finally, a truncated form of BmpB fused to maltose binding protein (MBP-F604) was investigated as a candidate for a vaccine.

Pigs and Immunisation Protocols

Thirty-six weaner pigs were divided into three groups. Group A were unvaccinated and housed in one pen in a room in an isolation animal house. Group B comprised 12 pigs immunised intramuscularly with 1 mg recombinant BmpB (30 kDa lipoprotein of B. hyodysenteriae) emulsified with 30% volume of adjuvant VSA3, in a total volume of 2 ml. Group C comprised 12 pigs immunised with 1 mg recombinant MBP-F604 (8 kDa C-terminal portion of BmpB fused to maltose-binding protein), again in VSA3.

Both vaccinated groups received a second intramuscular vaccination together with an oral boost (1 mg in a 40 ml volume of PBS, without adjuvant, by gastric intubation) 3 weeks after the 1st vaccination. Vaccinated groups B and C were housed in separate pens in the same isolation room. All 36 pigs were challenged orally with 50 ml of exponential log-phase (−10⁸/ml) Australian B. hyodysenteriae strain “Brentwood/Q02”, using a stomach tube., The inoculum was given daily for 5 consecutive days, starting two weeks after the oral vaccination.

Sampling and Post-Mortem

Blood samples were collected from the jugular vein prior to the first vaccination, just prior to the second vaccination, prior to the first day of challenge, and at necropsy. Sera were collected using standard techniques and tested in ELISA for systemic antibodies to the vaccine antigen, and also in Western Blot analysis against cellular extracts of B. hyodysenteriae.

Rectal faeces from all pigs were collected three times per week and the swabs cultured. When dysentery was observed (fresh blood and mucus in the faeces), pigs were immediately removed for necropsy. All other pigs which did not develop diarrhoea were killed and necropsied 51 days after experimental challenge. The presence of gross lesions along the large intestine was recorded. Caecal swabs were cultured for spirochaetes. Colonic scrapings were collected and tested for specific immunoglobulin content by ELISA and Western blot analysis.

Spirochaetal Culture

Swabs were streaked onto trypticase soy agar plates containing 5% (v/v) defibrinated sheep blood, spectinomycin (400 μg/ml), colistin (25 μg/ml) and vancomycin (25 μg/ml). Plates were incubated at 37° C. in an anaerobic environment for seven days. Spirochaetes were identified as B. hyodysenteriae on the basis of strong beta-haemolysis, microscopic morphology and NADH oxidase (nox) PCR of cell growth on the plates.

ELISA (Serum)

Wells on Microtitre plates (Immulon 4HBX, Dynex) were coated with an aliquot (100 μl) of either purified BmpB (0.5 ug/ml), purified MBP-F604 (1 μg/ml) or whole-cell extract of B. hyodysenteriae (1 μg/ml) in carbonate buffer (pH 9.6). Plates were incubated overnight at 4° C.

A blocking solution (150 μl) of PBS-BSA (1% w/v)) was added to the wells of the plates and the plates incubated for 1 hour at room temperature, with mixing and then washed three times with 150 μl of PBST (0.05% v/v).

Samples of pig sera were diluted 1:200 in PBST-BSA (0.1% w/v) and the diluted samples (100 μl) added to the wells of the plates. The plates were incubated at room temperature for 2 hours, with mixing. Plates were then washed (as above) before adding an aliquot (100 μl) of goat anti-swine IgG (whole molecule)HRP diluted 1:5,000 in PBST and incubated for 1 hr at room temperature. The plates were washed and 100 μl of TMB substrate added.

Colour development was stopped after 10 minutes incubation at room temperature by the addition of 1 M sulphuric acid (50 μl). The optical density of each well was read at 450 nm using a micro-plate reader (Biorad Model 3550UV).

ELISA (Mucosal)

Scrapings were taken from a 5 cm section of the colon. The scrapings were resuspended in 1 ml of PBS containing 1% (w/v) BSA, 2 mM PMSF, 1 mM EDTA and 0.2% (w/v) sodium azide. Suspensions were mixed by vortex and centrifuged at 14,000 rpm for 10 minutes. The supernatant was removed, diluted 1:2 with PBST, and an aliquot (100 μl) used for ELISA.

The ELISA plates were coated with recombinant BMpB as indicated for the serum ELISA. The diluted colonic extracts were reacted with the coated antigen for 2 hours at Room temperature and then incubated for 1 hour with unconjugated rabbit anti-swine IgA (1:2,000) immunoglobulin. Bound anti-swine IgA antibody was detected using goat anti-rabbit IgG (whole molecule)HRP diluted 2000-fold. After incubating for 1 hour at room temperature, the plates were washed and an aliquot (100 μl) of TMB substrate added.

Colour development was stopped after 10 minutes incubation at room temperature by the addition of 1M sulphuric acid (50 μl). The optical density of each well was read at 450 nm using a micro-plate reader (Biorad Model 3550UV).

Western Blot Analysis

A sample of sonicated and cleared B. hyodysenteriae cell suspension (50 μg) was loaded onto a 10% (w/v) SDS-PAGE gel and the proteins were separated via electrophoresis under standard conditions. The separated proteins were then electro-transferred to a nitrocellulose membrane using a Biorad Mini Trans-blot cell under standard conditions. The membrane was then blocked with TBS-skim milk (5% w/v) and assembled into the multi-probe apparatus (Biorad). Samples of either 100 μl of diluted pooled pig serum (1:10) or mucosal supernatant (1:2) were added to the lanes of the multi-probe apparatus and incubated for 2 hours at room temperature. The lanes of the multi-probe apparatus were washed three times with TBST (0.1% v/v) to remove excess primary antibody.

For the serum antibody, 100 μl of goat anti-swine IgG-HRP (1:2,000) was added to each lane and incubated for 1 hour at room temperature. For mucosal antibody, 100 μl of rabbit anti-swine IgA (1:2,000) was added to each lane and incubated for 1 hour at room temperature, followed by a 1 hour incubation with 100 μl of goat anti-rabbit IgG-HRP (1:2,000).

The membrane was removed from the apparatus and washed three times with TBST. Colour development occurred in 10 ml of DAB solution (5 mg/ml, 0.0003% v/v hydrogen peroxide, TBS) and the membrane was washed with tap water when sufficient development had occurred. The membrane was dried and scanned for presentation.

Disease/Lesion Scoring

To allow numerical comparisons between the groups, an artificial scoring mechanism was devised as outlined in the following Table 8.

TABLE 8

| Score | Characteristics |
|---|---|
| 4 | severe lesions at post-mortem with clinical signs |
| 3 | mild colitis lesions at post-mortem with clinical signs |
| 2 | severe colitis at post-mortem with no clinical signs |
| 1 | mild lesions at post-mortem with no clinical signs |
| 0 | no clinical signs |

Results and Discussion

Serological Response to the Vaccination

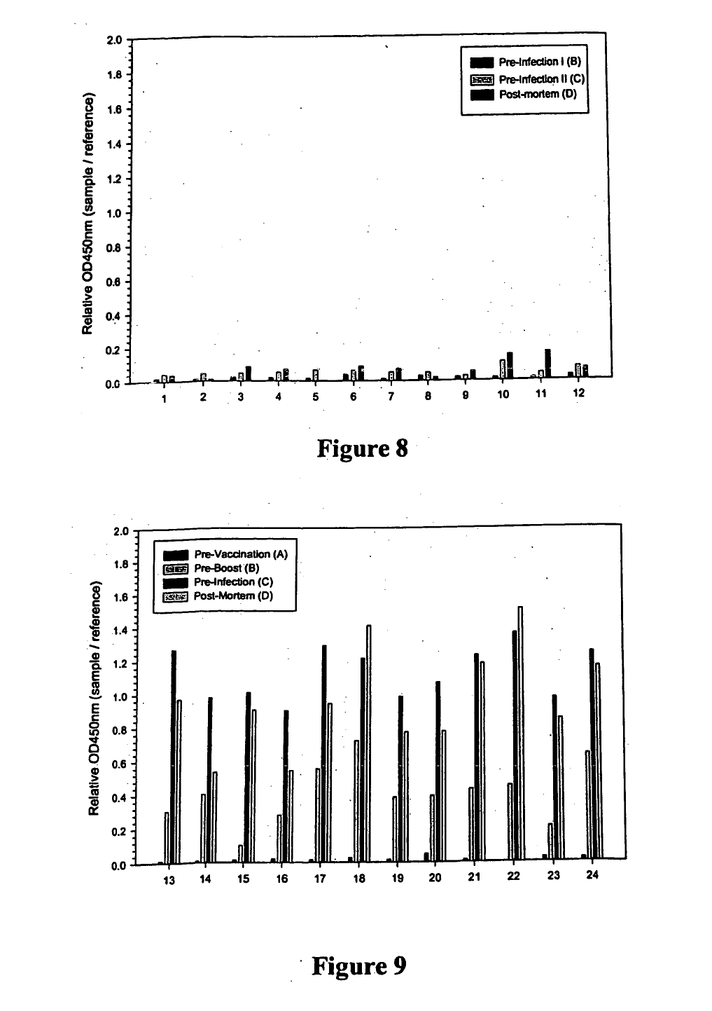

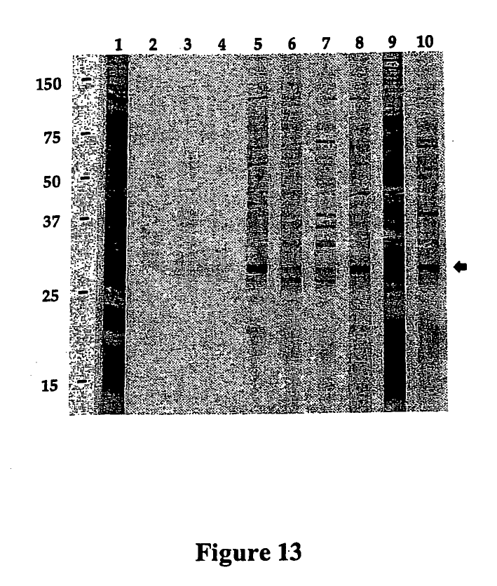

The systemic antibody response of the pigs to vaccination and challenge with B. hyodysenteriae are shown in FIGS. 8 to 11. Western Blot analysis of the vaccinated pigs against the whole-cell of B. hyodysenteriae is shown in FIGS. 12 and 13.

The unvaccinated pigs (Group A) showed negligible response to recombinant BmpB throughout the experimental period, although two pigs (pigs 10 and 11) developed a very slight titre following experimental challenge (FIG. 8). Both developed severe SD prior to the end of the experiment, and were removed. Pigs 2, 3, 5 and 12 from the non-vaccinated group also developed clinical SD, however they did not show an increase in systemic antibody titres to recombinant BmpB. None of the unvaccinated pigs showed detectable reactivity to the whole-cell of B. hyodysenteriae in Western Blot analysis (data not shown).

The pigs vaccinated with recombinant BmpB (Group B) responded strongly against BmpB following vaccination and oral boost (FIG. 9). Iwo pigs (pigs 18 and 22) showed a slight increase in systemic antibody titres following experimental challenge. Both developed clinical signs of SD and had mild lesions in the colon at post-mortem. The remaining pigs all showed a decrease in titres post-infection. These pigs did not develop clinical signs of SD, although pigs 14, 16 and 19 had mild to severe lesions in the colon at post-mortem.

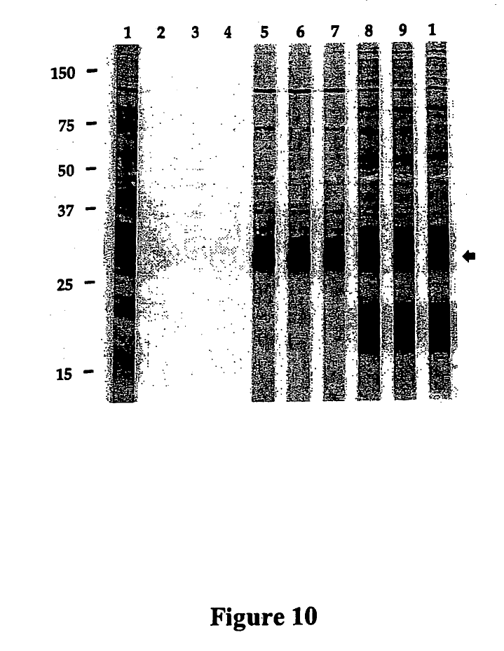

Western Blot analysis of pooled serum from the pigs of group B vaccinated with recombinant BmpB is shown in FIG. 10. Sera from four pigs were pooled for each sampling time. The antigen used was a whole-cell extract of the homologous B. hyodysenteriae strain used for challenge. This western blot analysis of serum from the BmpB vaccinated pigs indicated that the antibody response induced by the vaccination was directed at the native BmpB of B. hyodysenteriae used for challenge, although other bands were also observed.

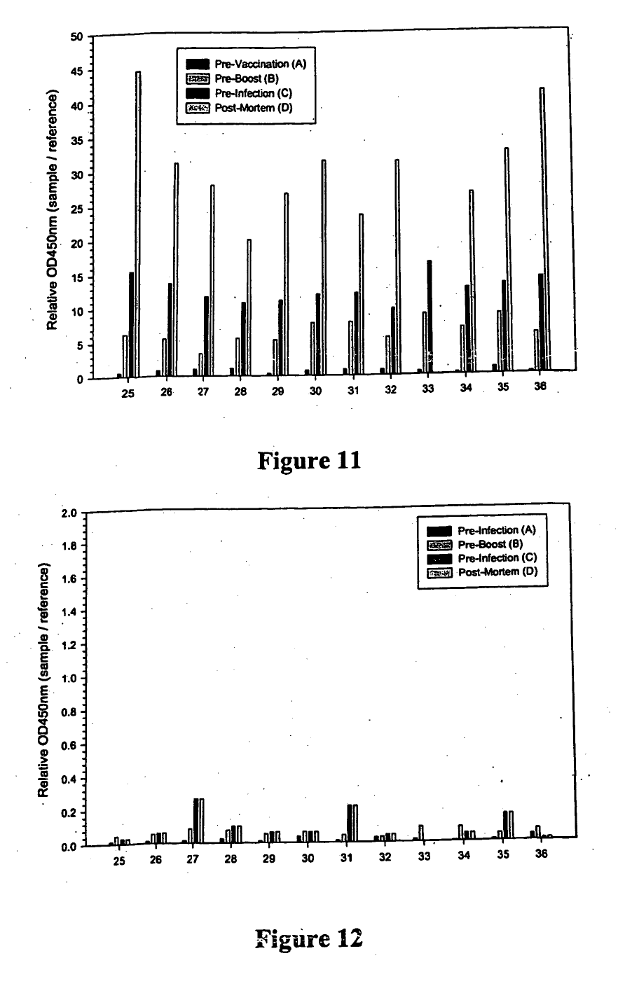

Pigs vaccinated with MBP-F604 (Group C) developed antibody titres to the vaccine component (FIG. 11). Following experimental challenge with B. hyodysenteriae, the systemic antibody titres of these pigs continued to increase, presumably as a response to the spirochaetal challenge.

However, the systemic titres induced by the MBP-F604 appear to have been directed mainly towards the MBP component of the vaccine, as these pigs only developed slight titres against recombinant BmpB (FIG. 12). Pigs 27, 31 and 35 developed a slightly higher antibody response towards recombinant BmpB than the others pigs in this group. Pig 27 showed clinical signs of SD and had severe lesions in the colon at post-mortem. Pig 31 and 35 did not develop clinical signs of SD, but pig 31 had extensive lesions in the colon at post-mortem.

Western Blot analysis of pooled serum from the pigs of group C that were vaccinated against MBP-F604 is shown in FIG. 13. Sera from three pigs which indicated some ELISA reactivity to recombinant BmpB was investigated. The antigen used was a whole cell extract of the homologous B. hyodysenteriae strain used for challenge.

The western blot analysis of serum from pigs 27, 31 and 35 indicated that the antibody response induced by the vaccination in these three animals was directed against the native BmpB of B. hyodysenteriae.

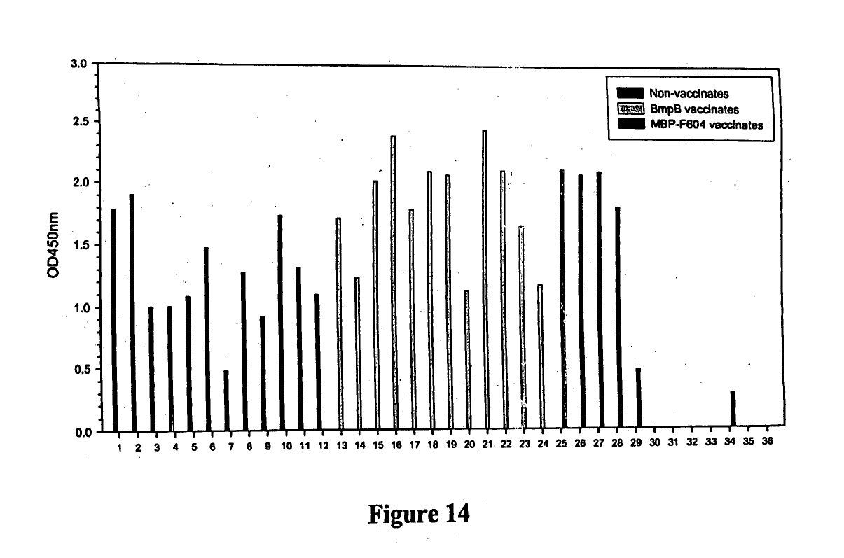

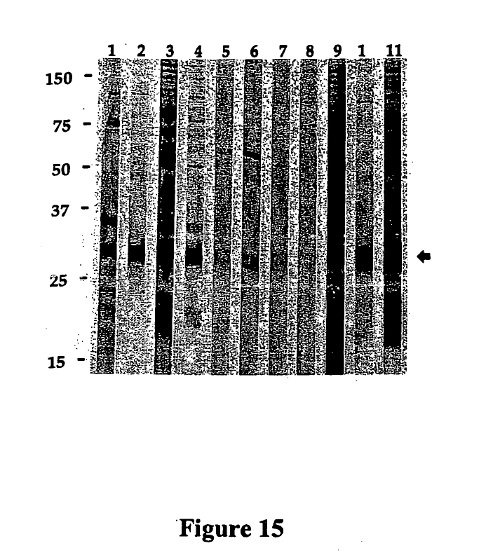

The local (colonic) IgA antibody response of all pigs to-the recombinant BmpB following vaccination and challenge is shown in FIG. 14. All unvaccinated pigs and pigs vaccinated with recombinant BmpB developed a local response to recombinant BmpB, with the latter group tending to have higher titres. Six of the twelve pigs (25-29, 34) vaccinated with MBP-F604 developed a local response to recombinant BmpB. Of the other six, one died of unknown causes, and one did not develop signs or have lesions in the colon—whilst the other four did. Western Blot analysis of selected pigs from each experimental group indicates that a proportion of the local response seen at the colon was directed at the native BmpB lipoprotein (FIG. 15). No correlation could be made relating local response and the severity of disease in these pigs. The local response also indicated that the presence of IgA antibodies directed against BmpB at the colon did not provide complete protection from clinical SD.

Brachyspira hyodysenteriae Excretion in the Faeces

The pattern of faecal excretion detected in pigs from the three groups is shown in Tables 9 to 11, respectively.

Table 9 shows individual colonization results for the unvaccinated pigs (Group A) after oral challenge with of B. hyodysenteriae. Colonisation was determined by culture of faecal swabs and PCR on growth plates. The date represents the day post-infection. The (−) symbol represents culture negative, (+) represents culture positive and (↓) indicates that no culture result was available as the pig had been removed for post mortem. For the unvaccinated pigs, excretion of B. hyodysenteriae was first detected in one pig (5) eight days after the end of experimental infection. Five pigs were killed before the end of the experiment (diarrhoea was observed and lesions were found, in the colon at post-mortem). The remaining seven pigs all had positive faecal cultures at some point during the experimental penrod. Pig 12 had clinical signs on the day of slaughter at the end of the experiment. The appearance of clinical signs of SD in all pigs was always preceded by the presence of positive faecal cultures.

TABLE 9

| Pig | Day | Day | Day | Day | Day | Day | Day | Day | Day | Day | Day | Day | Day | Day | Day | Day | Day | Day |
|---|---|---|---|---|---|---|---|---|---|---|---|---|---|---|---|---|---|---|
| Number | −4 | 3 | 6 | 8 | 10 | 14 | 17 | 21 | 22 | 24 | 27 | 29 | 31 | 34 | 37 | 42 | 44 | 51 |
| 1 | − | − | − | − | − | − | − | − | − | − | − | − | − | − | + | − | + | + |
| 2 | − | − | − | − | + | + | ↓ | ↓ | ↓ | ↓ | ↓ | ↓ | ↓ | ↓ | ↓ | ↓ | ↓ | ↓ |
| 3 | − | − | − | − | − | − | − | − | − | − | − | − | + | + | + | ↓ | ↓ | ↓ |
| 4 | − | − | − | − | − | − | − | − | − | − | − | − | + | − | − | − | − | + |
| 5 | − | − | − | + | + | ↓ | ↓ | ↓ | ↓ | ↓ | ↓ | ↓ | ↓ | ↓ | ↓ | ↓ | ↓ | ↓ |
| 6 | − | − | − | − | − | − | − | − | − | − | − | − | − | − | + | − | + | − |
| 7 | − | − | − | − | − | − | − | − | − | + | − | − | − | − | − | − | − | − |
| 8 | − | − | − | − | − | − | − | − | − | − | − | − | − | − | − | − | + | − |
| 9 | − | − | − | − | − | − | − | − | − | − | − | − | − | − | − | + | + | + |
| 10 | − | − | − | − | + | + | + | ↓ | ↓ | ↓ | ↓ | ↓ | ↓ | ↓ | ↓ | ↓ | ↓ | ↓ |
| 11 | − | − | − | − | − | − | − | + | − | − | + | − | + | + | + | ↓ | ↓ | ↓ |
| 12 | − | − | − | − | − | − | − | − | − | − | − | − | − | − | + | + | − | + |
| % | 0 | 0 | 0 | 8.3 | 25 | 18.2 | 10 | 11.1 | 0 | 11.1 | 11.1 | 0 | 33.3 | 22.2 | 55.6 | 28.6 | 57.1 | 57.1 |
| Culture | (0/ | (0/ | (0/12) | (1/12) | (3/12) | (2/11) | (1/10) | (1/9) | (0/9) | (1/9) | (1/9) | (0/9) | (3/9) | (2/9) | (5/9) | (2/7) | (4/7) | (4/7) |
| Positive | 12) | 12) | | | | | | | | | | | | | | | | |

Table 10 shows individual colonization results for the vaccinated pigs (Group B) after oral challenge with of B. hyodysenteriae. Colonisation was determined by culture of faecal swabs and PCR on growth plates. The date represents the day post-infection. The (−) symbol represents culture negative, (+) represents culture positive and (↓) indicates that no culture result was available as the pig had been removed for post mortem. For the pigs vaccinated with BmpB, the first faecal positive pig (23) was detected ten days after experimental infection, although ft did not go on to develop clinical signs. Two pigs (18 and 22) were killed before the end of the experiment due to the presence of diarrhoea, and subsequently lesions were found in their colons at post-mortem (although pig 22 was culture negative at post-mortem). Of the remaining ten pigs, nine were faecal positive at some point, but only two of these nine were culture positive at post-mortem.

Table 11 shows individual colonization results for the vaccinated pigs (Group C) after oral challenge with of B. hyodysenteriae. Colonisation was determined by culture of faecal swabs and PCR on growth plates. The date represents the day post-infection. The (−) symbol represents culture negative, (+) represents culture positive and (↓) indicates that no culture result was available as the pig had been removed for post mortem.

For the pigs vaccinated with MBP-F604, the first pig (25) became culture positive six days post-infection. Nine pigs were killed before the end of the experiment due to the presence of dysentery, and they had extensive and severe lesions in the colon at post-mortem. One pig (33) died due to an unknown cause not related to SD. Of the remaining two pigs, one (31) was frequently faecal positive during the experimental period, and extensive lesions were found in the colon at post-mortem. This pig was culture negative from the caecum. The other pig (35) remained faecal negative, and no lesions were found at post-mortem.

TABLE 10

| Pig | Day | Day | Day | Day | Day | Day | Day | Day | Day | Day | Day | Day | Day | Day | Day | Day | Day | Day |
|---|---|---|---|---|---|---|---|---|---|---|---|---|---|---|---|---|---|---|
| Number | −4 | 3 | 6 | 8 | 10 | 14 | 17 | 21 | 22 | 24 | 27 | 29 | 31 | 34 | 37 | 42 | 44 | 51 |
| 13 | − | − | − | − | − | − | − | + | + | + | + | + | + | − | + | − | − | − |
| 14 | − | − | − | − | − | − | − | − | + | + | + | + | + | − | − | − | + | − |
| 15 | − | − | − | − | − | − | − | − | − | − | − | − | − | − | − | − | − | − |
| 16 | − | − | − | − | − | − | − | − | − | − | − | − | − | − | − | − | + | + |
| 17 | − | − | − | − | − | − | − | − | − | − | − | − | − | − | + | + | + | − |
| 18 | − | − | − | − | − | − | − | + | + | − | − | + | ↓ | ↓ | ↓ | ↓ | ↓ | ↓ |
| 19 | − | − | − | − | − | − | − | − | + | + | − | + | + | − | − | − | + | + |
| 20 | − | − | − | − | − | − | − | − | + | − | − | − | + | − | + | + | + | − |
| 21 | − | − | − | − | − | − | − | − | − | − | − | − | − | − | + | − | − | − |
| 22 | − | − | − | − | − | − | − | − | + | ↓ | ↓ | ↓ | ↓ | ↓ | ↓ | ↓ | ↓ | ↓ |
| 23 | − | − | − | − | + | + | − | − | − | − | − | − | + | − | − | − | − | − |
| 24 | − | − | − | − | − | + | + | + | − | − | + | − | − | − | + | + | + | − |
| % | 0 | 0 | 0 | 8.3 | 16.7 | 8.3 | 25 | 50 | 58 | 27.3 | 27.3 | 36.4 | 50 | 0 | 50 | 30 | 60 | 20 |
| Culture | (0/ | (0/ | (0/ | (1/ | (2/ | (1/ | (3/ | (6/ | (7/ | (3/ | (3/ | (4/ | (5/ | (0/10) | (5/10) | (3/10) | (6/10) | (2/10) |
| Positive | 12) | 12) | 12) | 12) | 12) | 12) | 12) | 12) | 12) | 11) | 11) | 11) | 10) | | | | | |

TABLE 11

| Pig | Day | Day | Day | Day | Day | Day | Day | Day | Day | Day | Day | Day | Day | Day | Day | Day | Day | Day |
|---|---|---|---|---|---|---|---|---|---|---|---|---|---|---|---|---|---|---|
| Number | −4 | 3 | 6 | 8 | 10 | 14 | 17 | 21 | 22 | 24 | 27 | 29 | 31 | 34 | 37 | 42 | 44 | 51 |
| 25 | − | − | + | + | + | ↓ | ↓ | ↓ | ↓ | ↓ | ↓ | ↓ | ↓ | ↓ | ↓ | ↓ | ↓ | ↓ |
| 26 | − | − | − | − | − | − | − | + | + | ↓ | ↓ | ↓ | ↓ | ↓ | ↓ | ↓ | ↓ | ↓ |
| 27 | − | − | − | − | − | − | − | + | + | ↓ | ↓ | ↓ | ↓ | ↓ | ↓ | ↓ | ↓ | ↓ |
| 28 | − | − | − | − | − | − | − | + | + | ↓ | ↓ | ↓ | ↓ | ↓ | ↓ | ↓ | ↓ | ↓ |
| 29 | − | − | − | − | − | − | − | − | − | − | − | + | + | + | + | ↓ | ↓ | ↓ |
| 30 | − | − | − | − | − | − | + | + | + | ↓ | ↓ | ↓ | ↓ | ↓ | ↓ | ↓ | ↓ | ↓ |
| 31 | − | − | − | − | − | + | + | + | − | + | − | + | + | − | + | − | − | − |
| 32 | − | − | − | − | − | − | − | − | + | + | ↓ | ↓ | ↓ | ↓ | ↓ | ↓ | ↓ | ↓ |
| 33 | − | − | − | − | − | − | − | − | * | * | * | * | * | * | * | * | * | * |
| 34 | − | − | − | − | − | − | − | + | + | + | ↓ | ↓ | ↓ | ↓ | ↓ | ↓ | ↓ | ↓ |
| 35 | − | − | − | − | − | − | − | − | − | − | − | − | − | − | − | − | − | − |
| 36 | − | − | − | − | − | − | − | + | 1 | 1 | ↓ | ↓ | ↓ | ↓ | ↓ | ↓ | ↓ | ↓ |
| % | 0 | 0 | 8.3 | 8.3 | 8.3 | 9.1 | 18.2 | 63.6 | 70 | 80 | 0 | 66.7 | 66.7 | 33.3 | 66.7 | 0 | 0 | 0 |
| Culture | (0/ | (0/ | (1/ | (1/ | (1/12) | (1/11) | (2/11) | (7/11) | (7/10) | (4/5) | (0/3) | (2/3) | (2/3) | (1/3) | (3/3) | (0/2) | (0/2) | (0/2) |
| Positive | 12) | 12) | 12) | 12) | | | | | | | | | | | | | | |

Body Weight

The mean and standard deviation of body weights (kg) in the three groups are presented in Table 12. There was no significant difference in body weight between groups. This was most likely the result of a large number of animals developing disease and being removed by three weeks post-infection.

TABLE 12

| | Day of | Day | Day | Day | Day | Day | Day | Day | Day | Day |
|---|---|---|---|---|---|---|---|---|---|---|
| Group | weaning | −4 | 3 | 10 | 17 | 24 | 31 | 37 | 45 | 51 |
| A | 5.8 | 12.0 | 1503 | 17.6 | 23.5 | 28.9 | 35.5 | 40.7 | 45.9 | 51.6 |
| | (0.4) | (1.5) | (1.7) | (2.0) | (3.3) | (3.9) | (3..2) | (3.7) | (4.3) | (5.1) |
| B | 5.9 | 10.8 | 14.2 | 17.7 | 22.5 | 27.6 | 32.1 | 37.8 | 42.0 | 47.0 |
| | (0.6) | (1.5) | (2.1) | (3.0) | (3.1) | (4.0) | (4.4) | (3.4) | (3.1) | (6.6) |
| C | 5.7 | 10.5 | 13.7 | 16.2 | 21.1 | 23.4 | 25.2 | 29.1 | 36.5 | 47.0 |
| | (0.6) | (2.3) | (3.3) | (3.6) | (4.8) | (6.7) | (8.9) | (10.1) | (16.3) | (16.3) |

Development of Disease and Lesions at Post-Mortem

Table 13 presents data of the signs of disease and severity of colonic lesions in the pigs at post-mortem (PM). Pigs 1-12 were unvaccinated (Group A), pigs 13-24 were vaccinated with BmpB (Group B) and pigs 25-36 were vaccinated with MBP-F604 (Group C). PM culture was taken from the caecum; DYS indicates observation of diarrhoea, and EOE indicates end of experiment (i.e. animal healthy). Pigs were scored according to clinical signs of disease-and the severity of lesions in the colon (see Table 8 for scoring system).

As mentioned above, the assignment of pigs from each experimental group into the “disease score” categories is shown in Table 8. A high score indicates severe disease, and a low score indicates mild disease. These scores help to rank the, three groups in relation to disease, with the pigs vaccinated with MBP-F604 showing the most frequent and severe disease (mean score 3.45), and pigs vaccinated with BmpB showing the least disease (mean, score 0.92).

In group A, six pigs (50%) developed clinical signs of SD. Five of these six had severe mucohaemorrhagic colitis lesions at post-mortem, whilst one (pig 2) had mild focal lesions of colitis at post-mortem (Table 13). Two pigs (1 and 9) did not develop clinical signs of SD, but had severe lesions in the colon at post-mortem. Although pig 1 was culture negative from the caecum, it had severe lesions. The other four pigs were healthy at slaughter, and were culture negative.

Twelve pigs were vaccinated with BmpB (Group B). Two of these pigs developed dysentery, and both had mild localised lesions in the colon at post-mortem. The remaining ten pigs stayed healthy and survived to the end of the experiment without developing diarrhoea. Of these ten pigs, two pigs (pigs 14 and 19) had severe colonic lesions and one pig (pig 16) had mild localised lesions limited to the proximal colon. Pig 14 was culture negative at post-mortem, but had delivered a positive faecal culture several sampling times before then.

The remaining seven pigs also appeared healthy at the time of slaughter, and did not have any evidence of colitis, although six had been faecal positive sometime during the experimental period. Two of these six pigs (pigs 0.20 and 24) were culture positive at post-mortem. Pig 15 remained healthy and culture negative throughout the experiment.

Twelve pigs were vaccinated with MBP-F604 (Group C). Nine pigs developed diarrhoea, and had severe lesions in the colon. These pigs were also culture positive at slaughter. One pig (pig 33) died before the end of the experiment due to an unknown cause not related to SD. Of the two other pigs, one pig (pig 31) had severe lesions in the distal colon and was culture positive at post-mortem. The other pig (pig 35) was healthy and culture negative at the time of post-mortem.

TABLE 13

| Non-vaccinated pigs (Group A) | | | | | BmpB vaccinated pigs (Group B) | | | | | MBP-F604 vaccinated pigs (Group C) | | | | |
|---|---|---|---|---|---|---|---|---|---|---|---|---|---|---|
| | Reason | PM | Lesion | | | Reason | PM | Lesion | | | Reason | PM | Severity of | |
| Pig | for PM | Culture | severity | Score | Pig | for PM | Culture | severity | Score | Pig | for PM | Culture | lesions | Score |
| 1 | EOE | − | Severe | 2 | 13 | EOE | − | — | 0 | 25 | DYS | + | Severe | 4 |
| 2 | DYS | + | Mild | 3 | 14 | EOE | − | Severe | 2 | 26 | DYS | + | Severe | 4 |
| 3 | DYS | + | Severe | 4 | 15 | EOE | − | — | 0 | 27 | DYS | + | Severe | 4 |
| 4 | EOE | − | — | 0 | 16 | EOE | + | Mild | 1 | 28 | DYS | + | Severe | 4 |
| 5 | DYS | + | Severe | 4 | 17 | EOE | − | — | 0 | 29 | DYS | + | Severe | 4 |
| 6 | EOE | − | — | 0 | 18 | DYS | + | Mild | 3 | 30 | DYS | + | Severe | 4 |
| 7 | EOE | − | — | 0 | 19 | EOE | + | Severe | 2 | 31 | EOE | − | Severe | 2 |
| 8 | EOE | − | — | 0 | 20 | EOE | + | — | 0 | 32 | DYS | + | Severe | 4 |
| 9 | EOE | + | Severe | 2 | 21 | EOE | − | — | 0 | 33 | UNKN | na | na | na |
| 10 | DYS | + | Severe | 4 | 22 | DYS | − | Mild | 3 | 34 | DYS | + | Severe | 4 |
| 11 | DYS | − | Severe | 4 | 23 | EOE | − | — | 0 | 35 | EOE | − | — | 0 |
| 12 | DYS | + | Severe | 4 | 24 | EOE | + | — | 0 | 36 | DYS | + | Severe | 4 |
| — | — | — | Mean | 2.25 | — | — | — | Mean | 0.92 | — | — | — | Mean | 3.45 |

### Conclusions

In summary, twelve unvaccinated control pigs (group A) were housed in a different room from the two pens of vaccinated pigs. Following experimental challenge with B. hyodysenteriae, six (50%) developed clinical signs of SD, and 2 additional pigs (16.7%) had evidence of severe colitis and spirochaetal colonisation at slaughter.

Pigs in group B were vaccinated twice intramuscularly (im) and once orally. (concurrent with the second im vaccination) with 1 mg BmpB in VSA3 adjuvant. They developed strong primary and secondary serological responses to BmpB. Following challenge, only two of the twelve pigs (16.7%) developed clinical signs of SD, and they only had mild colonic lesions at post-mortem. An additional three pigs (25%) were found to have colonic lesions at slaughter at the end of the experiment, although they did not show clinical signs. Two had quite extensive lesions, whilst one had mild localised lesions;

The pigs of group C were vaccinated with MBP-F604, following the same protocol as for BmpB. They developed good primary and secondary serological responses to MBP-F604, and this was boosted substantially following experimental infection. The pigs did not develop antibodies to BmpB. One pig died of unknown causes, whilst nine of the remaining 11 (81.8%) developed clinical signs of SD, in each case with severe colonic lesions at slaughter. One of the two remaining pigs (9%) had quite extensive colitis at the end of the experiment, despite appearing clinically unaffected.

Overall, the BmpB provided a relatively good level of protection from disease, especially if compared to the other vaccinated pigs in the same room. This confirms the previous findings above relating to BmpB. The VSA3 adjuvant appeared to act satisfactorily. The truncated fusion protein was immunogenic, but did not induce a protective response. In part, this may have been associated with the use of an MBP fusion, with the MBP perhaps physically impeding interactions between the immune system and the truncated protein.

Vaccination with BmpB provided relative but not complete protection against experimental SD. Fewer vaccinated pigs develop clinical signs than unvaccinated pigs, and there was less faecal shedding of the spirochaete. Vaccination with MBP-F604 was not protective.

It would be useful to examine the protection conferred by a product in which the truncated form of BmpB was fused to another smaller and more immunogenic carrier protein. This could be examined in mice, in the first instance. Small-scale field trials using BmpB for vaccination on a piggery with SD would also help to determine the likely potential of the antigen in field conditions.

1-56. (canceled)

57. An isolated polynucleotide comprising DNA having the sequence selected from the group consisting of
(a) SEQ ID NO: 1;
(b) a polynucleotide sequence that encodes a protein of SEQ ID NO: 2;
(c) a polynucleotide sequence that encodes for BmpB; and
(d) a polynucleotide sequence that is at least 80% homologous to the sequence of SEQ ID NO: 1 and binds to DNA encoding SEQ ID NO: 1 under stringent conditions.

58. A vector comprising the polynucleotide of claim 57.

59. The vector of claim 58 wherein said vector is an expression vector.

60. A cell containing the vector of claim 58.

61. A pharmaceutical composition comprising the vector of claim 58 and a pharmaceutically acceptable carrier.

62. A pharmaceutical composition comprising the polynucleotide of claim 57 and a pharmaceutically acceptable carrier.

63. A kit comprising a polynucleotide that is complementary to the polynucleotide of claim 57, a suitable container and instructions for its use.


SEQUENCE LISTING (25 sequences)

SEQ ID NO: 1 - DNA, 816 bp, Brachyspira hyodysenteriae
  promoter (173)...(178): putative promoter
atgaaaaaatttttattattggtatcatcagccatattatcattaatgatattatcatgcggaaatacttcttctggtgatcaaaagatagttaaagttggttttgctggagagtctgattatcaaatttgggatcctatagtagctaaattagctgaagaaggaattaaagtagagctagtatctttctctgattatactatacctaatcaggctttgaatgacggagaaattgacttgaatgcttttcagcattatgcatactttaatgatgaagtatcaaataaaggatatgacttaactgctattgctgatacttatatatctgctatgaatatttattctactaatattactgatgtaaaagaattaaaaaatggcgataaaatagctatacctaatgacccttctaatggaggaagagctttaaaagttcttcaggctgcaggaatcattaaagtaaaacctgaagcaggagatactcctagcgtaagcgatataataaaaaatcctctaaatattgaaatagtagaaatggatgcaggtgctatttacggtgttcttcctgatgttgcttgtgctgttatcaatggaaactatgctatatacttcggtttgaatcctggttctgattatatattcaaagatgatccttctatttacagcggaaaatcttttgttaatttaatagctgcaagaactaaagataaagataatgaattatacaaaaaagttgtagaaacttatcaatctgaaatagtagaaaaagtttataatgaaaatttcttaggttcttatcttcctacttggaaataa

SEQ ID NO: 2 - protein, 271 aa, Brachyspira hyodysenteriae
  SIGNAL (1)...(19)
MKKFLLLVSSAILSLMILSCGNTSSGDQKIVKVGFAGESDYQIWDPIVAKLAEEGIKVELVSFSDYTIPNQALNDGEIDLNAFQHYAYFNDEVSNKGYDLTAIADTYISAMNIYSTNITDVKELKNGDKIAIPNDPSNGGRALKVLQAAGIIKVKPEAGDTPSVSDIIKNPLNIEIVEMDAGAIYGVLPDVACAVINGNYAIYFGLNPGSDYIFKDDPSIYSGKSFVNLIAARTKDKDNELYKKVVETYQSEIVEKVYNENFLGSYLPTWK

SEQ ID NO: 3 - protein, 19 aa, Brachyspira hyodysenteriae
MKKFLLLVSSAILSLMILS

SEQ ID NO: 4 - protein, 17 aa, Brachyspira hyodysenteriae
  PEPTIDE (0)...(0): BmpB protein fragment
GAIYGVLPDVACAVING

SEQ ID NO: 5 - protein, 19 aa, Brachyspira hyodysenteriae
  PEPTIDE (0)...(0): BmpB protein fragment
FLLLVSSAILSLMILSCGN

SEQ ID NO: 6 - protein, 10 aa, Brachyspira hyodysenteriae
  PEPTIDE (0)...(0): BmpB protein fragment
KVELVSFSDY

SEQ ID NO: 7 - protein, 16 aa, Brachyspira hyodysenteriae
  PEPTIDE (0)...(0): BmpB protein fragment
ALKVLQAAGIIKVKPE

SEQ ID NO: 8 - protein, 9 aa, Brachyspira hyodysenteriae
  PEPTIDE (0)...(0): BmpB protein fragment
IWDPIVAKL

SEQ ID NO: 9 - protein, 8 aa, Brachyspira hyodysenteriae
  PEPTIDE (0)...(0): BmpB protein fragment
YKKVVETY

SEQ ID NO: 10 - protein, 9 aa, Brachyspira hyodysenteriae
  PEPTIDE (0)...(0): BmpB protein fragment
QKIVKVGFA

SEQ ID NO: 11 - protein, 15 aa, Brachyspira hyodysenteriae
  PEPTIDE (0)...(0): BmpB protein fragment
PSIYSGKSFVNLIAA

SEQ ID NO: 12 - protein, 6 aa, Brachyspira hyodysenteriae
  PEPTIDE (0)...(0): BmpB protein fragment
AIYFGL

SEQ ID NO: 13 - protein, 7 aa, Brachyspira hyodysenteriae
  PEPTIDE (0)...(0): BmpB protein fragment
SEIVEKV

SEQ ID NO: 14 - protein, 6 aa, Brachyspira hyodysenteriae
  PEPTIDE (0)...(0): BmpB protein fragment
LGSYLP

SEQ ID NO: 15 - protein, 9 aa, Brachyspira hyodysenteriae
  PEPTIDE (0)...(0): BmpB protein fragment
LNAFQHYAY

SEQ ID NO: 16 - protein, 7 aa, Brachyspira hyodysenteriae
  PEPTIDE (0)...(0): BmpB protein fragment
TPSVSDI

SEQ ID NO: 17 - protein, 9 aa, Brachyspira hyodysenteriae
  PEPTIDE (0)...(0): BmpB protein fragment
DLTAIADTY

SEQ ID NO: 18 - DNA, 29 bp, Artificial Sequence
  BmpB-F13-Xho1 primer
aaactcgagttattattggtatcatcagc

SEQ ID NO: 19 - DNA, 30 bp, Artificial Sequence
  BmpB-R195-EcoR1 primer
tatgaattcatcagagaaagatactagctc

SEQ ID NO: 20 - DNA, 30 bp, Artificial Sequence
  BmpB-R411-EcoR1 primer
tccgaattcagaagggtcattaggtatagc

SEQ ID NO: 21 - DNA, 30 bp, Artificial Sequence
  BmpB-R613-EcoR1 primer
gatgaattccgaagtatatagcatagtttc

SEQ ID NO: 22 - DNA, 29 bp, Artificial Sequence
  BmpB-R809-EcoR1 primer
tatgaattccaagtaggaagataagaacc

SEQ ID NO: 23 - DNA, 23 bp, Artificial Sequence
  pTrcHis-F primer
caatttatcagacaatctgtgtg

SEQ ID NO: 24 - DNA, 31 bp, Artificial Sequence
  BmpB-F604-Xho1 primer
aacctcgagatatacttcggtttgaatcctg

SEQ ID NO: 25 - DNA, 29 bp, Artificial Sequence
  BmpB-R809-EcoR1 primer
tatgaattccaagtaggaagataagaacc